(19) United States
(12) Patent Application Publication
(10) Pub. No.: US 2009/0131956 A1
(43) Pub. Date: May 21, 2009

(54) METHOD AND APPARATUS FOR PASSING SUTURE THROUGH THE LABRUM OF A HIP JOINT IN ORDER TO SECURE THE LABRUM TO THE ACETABULUM

(76) Inventors: Jonathan Dewey, Sunnyvale, CA (US); William Kaiser, San Jose, CA (US); Chris Pamichev, Cupertino, CA (US); Thanh-Nga Doan, San Jose, CA (US); Jennifer Doan, San Jose, CA (US)

Correspondence Address: Mark J. Pandiscio;Pandiscio & Pandiscio, P.C., 470 Totten Pond Road, Waltham, MA, 02451-1914, US

(21) Appl. No.: 12/268,369

(22) Filed: Nov. 10, 2008

Related U.S. Application Data
(60) Provisional application No. 61/002,361, filed on Nov. 8, 2007.

(51) Int. Cl. A61B 17/04

(52) U.S. Cl. 606/144

(57) ABSTRACT

A suture passer comprising:
a handle;
a shaft extending distally from the handle;
first and second jaw members mounted to the distal end of the shaft, the first jaw member having a suture support for supporting a length of suture;
a pitch adjustment mechanism for adjusting the pitch of the first and second jaw members relative to the shaft;
a lever mechanism for opening and closing the first and second jaw members relative to one another; and
a needle mechanism for selectively urging a needle having a groove therein so that the groove in the needle can engage a length of suture supported by the suture support.

### REFERENCE TO PENDING PRIOR PATENT APPLICATION

This patent application claims benefit of pending prior U.S. Provisional Patent Application Ser. No. 61/002,361, filed Nov. 8, 2007 by Chris Pamichev for METHOD AND APPARATUS FOR PASSING SUTURE THROUGH THE LABRUM OF A HIP JOINT SO AS TO FACILITATE SECURING THE LABRUM TO THE ACETABULUM (Attorney's Docket No. FIAN-13 PROV), which patent application is hereby incorporated herein by reference.

### FIELD OF THE INVENTION

This invention relates to surgical methods and apparatus in general, and more particularly to methods and apparatus for treating the hip joint.

### BACKGROUND OF THE INVENTION

### The Hip Joint in General

The hip joint is a ball-and-socket joint which movably connects the leg to the torso. The hip joint is capable of a wide range of different motions, e.g., flexion and extension, abduction and adduction, medial and lateral rotation, etc. See FIGS. 1A, 1B, 1C and 1D.

With the possible exception of the shoulder joint, the hip joint is perhaps the most mobile joint in the body. Significantly, and unlike the shoulder joint, the hip joint carries substantial weight loads during most of the day, in both static (e.g., standing and sitting) and dynamic (e.g., walking and running) conditions.

The hip joint is susceptible to a number of different pathologies. These pathologies can have both congenital and injury-related origins. In some cases, the pathology can be substantial at the outset. In other cases, the pathology may be minor at the outset but, if left untreated, may worsen over time. More particularly, in many cases, an existing pathology may be exacerbated by the dynamic nature of the hip joint and the substantial weight loads imposed on the hip joint.

The pathology may, either initially or thereafter, significantly interfere with patient comfort and lifestyle. In some cases, the pathology can be so severe as to require partial or total hip replacement. A number of procedures have been developed for treating hip pathologies short of partial or total hip replacement, but these procedures are generally limited in scope due to the significant difficulties associated with treating the hip joint.

A better understanding of various hip joint pathologies, and also the current limitations associated with their treatment, can be gained from a more thorough understanding of the anatomy of the hip joint.

### Anatomy of the Hip Joint

The hip joint is formed at the junction of the femur and the hip. More particularly, and looking now at FIG. 2, the head of the femur is received in the acetabular cup of the hip, with a plurality of ligaments and other soft tissue serving to hold the bones in articulating condition.

More particularly, and looking now at FIG. 3, the femur is generally characterized by an elongated body terminating, at its top end, in an angled neck which supports a hemispherical head (also sometimes referred to as “the ball”). As seen in FIGS. 3 and 4, a large projection known as the greater trochanter protrudes laterally and posteriorly from the elongated body adjacent to the neck of the femur. A second, somewhat smaller projection known as the lesser trochanter protrudes medially and posteriorly from the elongated body adjacent to the neck. An intertrochanteric crest (FIGS. 3 and 4) extends along the periphery of the femur, between the greater trochanter and the lesser trochanter.

Looking next at FIG. 5, the hip socket is made up of three constituent bones: the ilium, the ischium and the pubis. These three bones cooperate with one another (they typically ossify into a single “hip bone” structure by the age of 25) so as to collectively form the acetabular cup. The acetabular cup receives the head of the femur.

Both the head of the femur and the acetabular cup are covered with a layer of articular cartilage which protects the underlying bone and facilitates motion. See FIG. 6.

Various ligaments and soft tissue serve to hold the ball of the femur in place within the acetabular cup. More particularly, and looking now at FIGS. 7 and 8, the ligamentum teres extends between the ball of the femur and the base of the acetabular cup. As seen in FIG. 9, a labrum is disposed about the perimeter of the acetabular cup. The labrum serves to increase the depth of the acetabular cup and effectively establishes a suction seal between the ball of the femur and the rim of the acetabular cup, thereby helping to hold the head of the femur in the acetabular cup. In addition to the foregoing, and looking now at FIG. 10, a fibrous capsule extends between the neck of the femur and the rim of the acetabular cup, effectively sealing off the ball-and-socket members of the hip joint from the remainder of the body. The foregoing structures (i.e., the ligamentum teres, the labrum and the fibrous capsule) are encompassed and reinforced by a set of three main ligaments (i.e., the iliofemoral ligament, the ischiofemoral ligament and the pubofemoral ligament) which extend between the femur and the perimeter of the hip socket. See, for example, FIGS. 11 and 12 which show the iliofemoral ligament, wherein FIG. 11 is an anterior view and FIG. 12 is a posterior view.

### Pathologies of the Hip Joint

As noted above, the hip joint is susceptible to a number of different pathologies. These pathologies can have both congenital and injury-related origins.

By way of example but not limitation, one important type of congenital pathology of the hip joint involves impingement between the neck of the femur and the rim of the acetabular cup. In some cases, and looking now at FIG. 13, this impingement can occur due to irregularities in the geometry of the femur. This type of impingement is sometimes referred to as a cam-type femoroacetabular impingement (i.e., a cam-type FAI). In other cases, and looking now at FIG. 14, the impingement can occur due to irregularities in the geometry of the acetabular cup. This latter type of impingement is sometimes referred to as a pincer-type femoroacetabular impingement (i.e., a pincer-type FAI). Impingement can result in a reduced range of motion, substantial pain and, in some cases, significant deterioration of the hip joint.

By way of further example but not limitation, another important type of congenital pathology of the hip joint involves defects in the articular surface of the ball and/or the articular surface of the acetabular cup. Defects of this type sometimes start fairly small but often increase in size over time, generally due to the dynamic nature of the hip joint and also due to the weight-bearing nature of the hip joint. Articular defects can result in substantial pain, induce and/or exacerbate arthritic conditions and, in some cases, cause significant deterioration of the hip joint.

By way of further example but not limitation, one important type of injury-related pathology of the hip joint involves trauma to the labrum. More particularly, in many cases, an accident or sports-related injury can result in the labrum being torn away from the rim of the acetabular cup, typically with a tear running through the body of the labrum. See FIG. 15. These types of injuries can be very painful for the patient and, if left untreated, can lead to substantial deterioration of the hip joint.

### The General Trend Toward Treating Joint Pathologies Using Minimally-Invasive, and Earlier, Interventions

The current trend in orthopedic surgery is to treat joint pathologies using minimally-invasive techniques. Such minimally-invasive, “keyhole” surgeries generally offer numerous advantages over traditional, “open” surgeries, including reduced trauma to tissue, less pain for the patient, faster recuperation times, etc.

By way of example but not limitation, it is common to re-attach ligaments in the shoulder joint using minimally-invasive, “keyhole” techniques which do not require laying open the capsule of the shoulder joint. By way of further example but not limitation, it is common to repair torn meniscal cartilage in the knee joint, and/or to replace ruptured ACL ligaments in the knee joint, using minimally-invasive techniques.

While such minimally-invasive approaches can require additional training on the part of the surgeon, such procedures generally offer substantial advantages for the patient and have now become the standard of care for many shoulder joint and knee joint pathologies.

In addition to the foregoing, in view of the inherent advantages and widespread availability of minimally-invasive approaches for treating pathologies of the shoulder joint and knee joint, the current trend is to provide such treatment much earlier in the lifecycle of the pathology, so as to address patient pain as soon as possible and so as to minimize any exacerbation of the pathology itself. This is in marked contrast to traditional surgical practices, which have generally dictated postponing surgical procedures as long as possible so as to spare the patient from the substantial trauma generally associated with invasive surgery.

### Treatment For Pathologies of the Hip Joint

Unfortunately, minimally-invasive treatments for pathologies of the hip joint have lagged far behind minimally-invasive treatments for pathologies of the shoulder joint and knee joint. This is generally due to (i) the constrained geometry of the hip joint itself, and (ii) the nature and location of the pathologies which must typically be addressed in the hip joint.

More particularly, the hip joint is generally considered to be a “tight” joint, in the sense that there is relatively little room to maneuver within the confines of the joint itself. This is in marked contrast to the shoulder joint and the knee joint, which are generally considered to be relatively “spacious” joints (at least when compared to the hip joint). As a result, it is relatively difficult for surgeons to perform minimally-invasive procedures on the hip joint.

Furthermore, the pathways for entering the interior of the hip joint (i.e., the pathways which exist between adjacent bones) are generally much more constraining for the hip joint than for the shoulder joint or the knee joint. This limited access further complicates effectively performing minimally-invasive procedures on the hip joint.

In addition to the foregoing, the nature and location of the pathologies of the hip joint also complicate performing minimally-invasive procedures on the hip joint. By way of example but not limitation, consider a typical detachment of the labrum in the hip joint. In this situation, instruments must generally be introduced into the joint space using an angle of approach which is offset from the angle at which the instrument addresses the tissue. This makes drilling into bone, for example, significantly more complicated than where the angle of approach is effectively aligned with the angle at which the instrument addresses the tissue, such as is frequently the case in the shoulder joint. Furthermore, the working space within the hip joint is typically extremely limited, further complicating repairs where the angle of approach is not aligned with the angle at which the instrument addresses the tissue.

As a result of the foregoing, minimally-invasive hip joint procedures are still relatively difficult to perform and relatively uncommon in practice. Consequently, patients are typically forced to manage their hip pain for as long as possible, until a resurfacing procedure or a partial or total hip replacement procedure can no longer be avoided. These procedures are generally then performed as a highly-invasive, open procedure, with all of the disadvantages associated with highly-invasive, open procedures.

As a result, there is, in general, a pressing need for improved methods and apparatus for treating pathologies of the hip joint.

### Re-attaching the Labrum of the Hip Joint

As noted above, hip arthroscopy is becoming increasingly more common in the diagnosis and treatment of various hip pathologies. However, due to the anatomy of the hip joint and the pathologies associated with the same, hip arthroscopy is currently practical for only selected pathologies and, even then, hip arthroscopy has generally met with limited success.

One procedure which is sometimes attempted arthroscopically relates to the repair of a torn and/or detached labrum. This procedure may be attempted (i) when the labrum has been damaged but is still sufficiently healthy and intact as to be capable of repair and/or re-attachment, and (ii) when the labrum has been deliberately detached (e.g., so as to allow for acetabular rim trimming to treat a pathology such as a pincer-type FAI) and needs to be subsequently re-attached. See, for example, FIG. 16, which shows a normal labrum which has its base securely attached to the acetabular cup, and FIG. 17, which shows a portion of the labrum (in this case the tip) detached from the acetabular cup. In this respect it should also be appreciated that repairing the labrum rather than removing the labrum is generally desirable, inasmuch as studies have shown that patients whose labrum has been repaired tend to have better long-term outcomes than patients whose labrum has been removed.

Unfortunately, current methods and apparatus for arthroscopically re-attaching the labrum are somewhat problematic. The present invention is intended to improve upon the current approaches for labrum re-attachment.

More particularly, current approaches for arthroscopically re-attaching the labrum typically use apparatus originally designed for use in re-attaching ligaments to bone. For example, one such approach utilizes a screw-type bone anchor, with two sutures extending therefrom, and involves deploying the bone anchor in the acetabulum above the labrum re-attachment site. A first one of the sutures is passed either through the detached labrum or, alternatively, around the detached labrum. Then the first suture is tied to the second suture so as to support the labrum against the acetabular rim.

Unfortunately, it can be difficult to arthroscopically pass suture through the labrum in a manner which facilitates re-attaching the labrum to the acetabulum. This is due to space limitations within the hip joint, the angle of approach into the hip joint, the nature of the labral tissue, the position of the labrum within the hip joint, etc.

More particularly, the labrum is a relatively thin structure which normally lines the outer portion of the acetabular cup, with the tip of the labrum extending up and over the rim of the acetabular cup (FIG. 16). In some ways, the labrum has a geometry which is somewhat similar to a layer of an onion: it has a large surface area but is relatively thin. This thinness presents a problem when passing suture through the labrum, since it is generally desirable to pass the suture through the labrum so that the suture does not open on the articular surface of the labrum, in order to prevent abrasion during joint motion. In other words, it is generally desirable to pass the suture through the labrum so that the suture extends within the depth of the labrum (i.e., parallel to the plane of the labrum) rather than through the face of the labrum (i.e., transverse to the front and back surfaces of the labrum). Unfortunately, current arthroscopic approaches for the repair of the labrum generally “lasso” or encircle the labrum with a loop of suture, which leaves a portion of the suture loop protruding through the articulating side of the labrum, where it may contact and abrade the articular cartilage on the head of the femur.

Another problem with current techniques for repairing the labrum relates to the anatomical position of the repair itself. More particularly, the bone anchor is typically deployed in the acetabular shelf, up “above” the rim of the acetabular cup. Such bone anchor placement is less than ideal, since it generally results in the labrum being drawn away from the joint, thereby complicating proper anatomical repair.

Furthermore, the labrum is made up of a large number of filaments arranged in a generally parallel configuration. Thus, in order to prevent the passed suture from pulling back through the labrum, it is generally desirable to pass the suture through the labrum so that there is a lateral offset between the suture's entry point and exit point. This approach ensures that the suture path crosses a plurality of filaments, whereby to resist pull-through. However, this can be difficult to achieve arthroscopically within the hip joint.

Accordingly, a primary object of the present invention is to provide a new approach for passing suture through the labrum so as to facilitate securing the labrum to the acetabulum, with the suture being placed in the anatomy so that it does not contact the articulating cartilage of the joint.

### SUMMARY OF THE INVENTION

The present invention provides a novel method and apparatus for arthroscopically passing suture through the labrum so as to facilitate securing the labrum to the acetabulum in an anatomically desirable manner.

Significantly, this new approach preferably passes the suture through the thickness of the labrum, from the tip of the labrum to the base of the labrum, so that the suture can be tied or otherwise secured on the extra-articular (i.e., capsular) side of the labrum, whereby to leave the articular side of the labrum free of suture.

In one preferred form of the invention, there is provided a suture passer comprising:

a handle;

a shaft extending distally from the handle;

first and second jaw members mounted to the distal end of the shaft, the first jaw member having a suture support for supporting a length of suture;

a pitch adjustment mechanism for adjusting the pitch of the first and second jaw members relative to the shaft;

a lever mechanism for opening and closing the first and second jaw members relative to one another; and

a needle mechanism for selectively urging a needle having a groove therein so that the groove in the needle can engage a length of suture supported by the suture support.

In another form of the invention, there is provided a method for passing suture through soft tissue, the method comprising the steps of:

providing a suture passer comprising:

a handle;

a shaft extending distally from the handle;

first and second jaw members mounted to the distal end of the shaft, the first jaw member having a suture support for supporting a length of suture;

a pitch adjustment mechanism for adjusting the pitch of the first and second jaw members relative to the shaft;

a lever mechanism for opening and closing the first and second jaw members relative to one another; and

a needle mechanism for selectively urging a needle having a groove therein so that the groove in the needle can engage a length of suture supported by the suture support;

positioning the needle so that it is withdrawn from the opening, positioning the first and second jaw members so that they are in their closed position, and positioning the suture in the suture support;

advancing the suture passer so that the first and second jaw members are adjacent to the tissue through which the suture is to be passed;

opening the first and second jaw members, adjusting the pitch of the first and second jaw members so that the first and second jaw members are aligned with the tissue, advancing the suture passer so that the first and second jaw members engage the tissue, and closing the first and second jaw members so that they securely grasp the tissue;

advancing the needle so that it passes through the tissue and the groove engages the suture; and

withdrawing the needle so that it passes out of the tissue, carrying the suture therewith, so that the suture is passed through the tissue.

In another form of the invention, there is provided a suture passer comprising:

a handle;

a shaft extending distally from the handle;

first and second jaw members pivotally mounted to the distal end of the shaft, the first jaw member having an opening formed therein and a suture support for supporting a length of suture adjacent the opening;

a slide movably mounted to the handle;

a lever arm for moving the slide relative to the handle;

first and second yokes movably disposed on the slide, and a yoke movement mechanism for selectively (i) urging first yoke distally and second yoke proximally, or (ii) urging first yoke proximally and second yoke distally;

a first control rod for connecting the first yoke to the first jaw member, and a second control rod for connecting the second yoke to the second jaw member;

a trigger pivotally mounted to the handle;

a needle slidably disposed within the shaft, the distal end of the needle having a groove therein and the proximal end of the needle being secured to the trigger so that the trigger can urge the needle through the opening in the first jaw member so that the groove in the needle can engage a length of suture supported by the suture support;

whereby (i) the pitch of the first and second jaw members relative to the longitudinal axis of the shaft may be adjusted via the yoke movement mechanism, (ii) the first and second jaw members may be opened and closed relative to one another via movement of the lever arm relative to the handle, and (iii) the needle can be advanced through the opening in the first jaw member so that the groove in the needle can engage a length of suture supported by the suture support via movement of the trigger relative to the handle.

In another form of the invention, there is provided a method for passing suture through soft tissue, the method comprising:

providing a suture passer comprising:
a handle;
a shaft extending distally from the handle;
first and second jaw members pivotally mounted to the distal end of the shaft, the first jaw member having an opening formed therein and a suture support for supporting a length of suture adjacent the opening;
a slide movably mounted to the handle;
a lever arm for moving the slide relative to the handle;
first and second yokes movably disposed on the slide, and a yoke movement mechanism for selectively (i) urging first yoke distally and second yoke proximally, or (ii) urging first yoke proximally and second yoke distally;
a first control rod for connecting the first yoke to the first jaw member, and a second control rod for connecting the second yoke to the second jaw member;
a trigger pivotally mounted to the handle;
a needle slidably disposed within the shaft, the distal end of the needle having a groove therein and the proximal end of the needle being secured to the trigger so that the trigger can urge the needle through the opening in the first jaw member so that the groove in the needle can engage a length of suture supported by the suture support;
whereby (i) the pitch of the first and second jaw members relative to the longitudinal axis of the shaft may be adjusted via the yoke movement mechanism, (ii) the first and second jaw members may be opened and closed relative to one another via movement of the lever arm relative to the handle, and (iii) the needle can be advanced through the opening in the first jaw member so that the groove in the needle can engage a length of suture supported by the suture support via movement of the trigger relative to the handle;

positioning the needle so that it is withdrawn from the opening, positioning the first and second jaw members in their closed position, and positioning the suture in the suture support;

advancing the suture passer so that the first and second jaw members are adjacent to the tissue through which the suture is to be passed;

opening the first and second jaw members, adjusting the pitch of the first and second jaw members so that the first and second jaw members are aligned with the tissue, advancing the suture passer so that the first and second jaw members engulf the tissue, and closing the first and second jaw members;

advancing the needle so that it passes through the tissue and through the opening so that the groove engages the suture; and

withdrawing the needle so that it passes out of the opening and out of the tissue, carrying the suture therewith, so that the suture is passed through the tissue.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIGS. 1A-1D are schematic views showing various aspects of hip motion;

FIG. 2 is a schematic view showing the bone structure in the region of the hip joints;

FIG. 3 is a schematic view of the femur;

FIG. 4 is a schematic view of the top end of the femur;

FIG. 5 is a schematic view of the pelvis;

FIGS. 6-12 are schematic views showing the bone and soft tissue structure of the hip joint;

FIG. 13 is a schematic view showing cam-type femoroacetabular impingement (FAI);

FIG. 14 is a schematic view showing pincer-type femoroacetabular impingement (FAI);

FIG. 15 is a schematic view showing a labral tear;

FIG. 16 is a schematic view showing the labrum attached to the acetabular cup;

FIG. 17 is a schematic view showing a portion of the labrum detached from the acetabular cup;

FIG. 18 is a schematic view of a novel suture passer formed in accordance with the present invention;

FIG. 19 is a schematic view showing the distal end of the novel suture passer shown in FIG. 18;

FIGS. 20-23 and 23A-23E are schematic views showing the upper and lower jaw members of the suture passer, and also an associated pitch adjustment mechanism for varying the pitch of the upper and lower jaw members relative to the longitudinal axis of the shaft of the suture passer;

FIGS. 24-27 are schematic views showing the upper and lower jaw members of the suture passer, and also an associated lever mechanism for opening and closing the upper and lower jaw members relative to one another;

FIGS. 28-32 are schematic views showing the upper and lower jaw members of the suture passer, and also an associated needle mechanism for selectively advancing and retracting a needle through tissue disposed between the upper and lower jaw members;

FIG. 32A is a schematic view showing further construction details for the upper and lower jaw members and their associated needle mechanism;

FIGS. 32B-32H are schematic views showing further construction details for the needle of the needle mechanism;

FIGS. 33-44 and 44A are schematic views showing how the suture passer of the present invention can be used to re-attach the labrum to the acetabulum during a procedure to address pincer-type impingement;

FIGS. 45-48 are schematic views showing additional constructions for the upper and lower jaw members of the suture passer;

FIGS. 49-51 are schematic views showing additional needle constructions;

FIGS. 52-55 and 55A are schematic views showing additional constructions for the upper and lower jaws members;

FIGS. 56-60 are schematic views showing how the suture passer may be equipped with a pair of needle mechanisms;

FIGS. 61-65 are schematic views showing additional constructions for the handle mechanism of the suture passer; and

FIG. 66 is a schematic view showing how the suture passer can be used to deliver a bone anchor to the acetabulum.

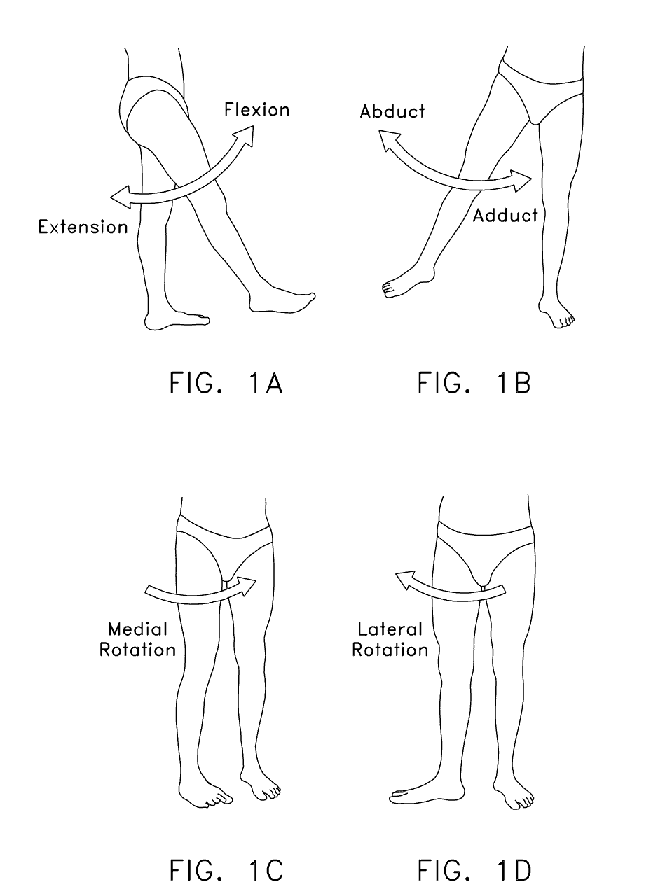

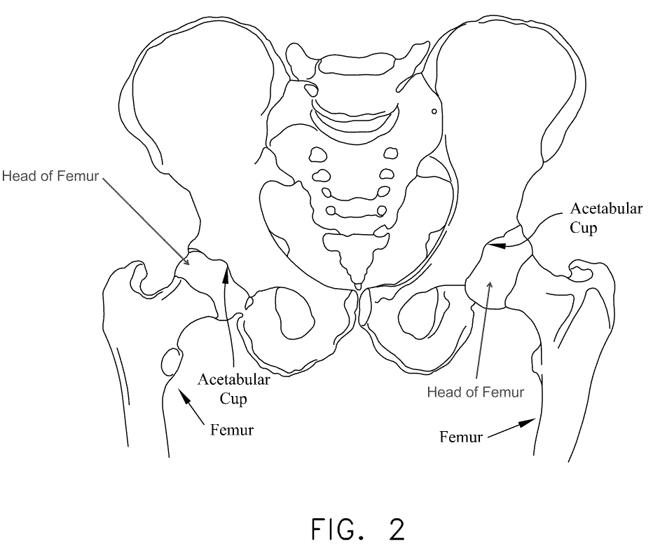

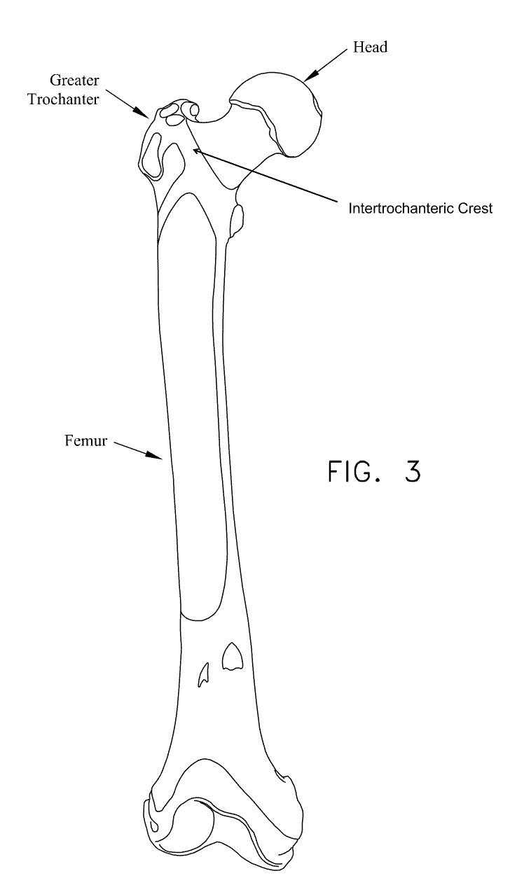

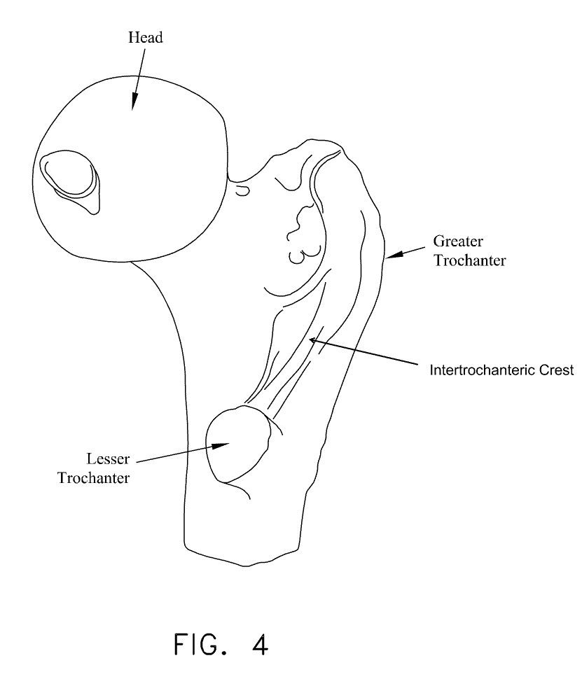

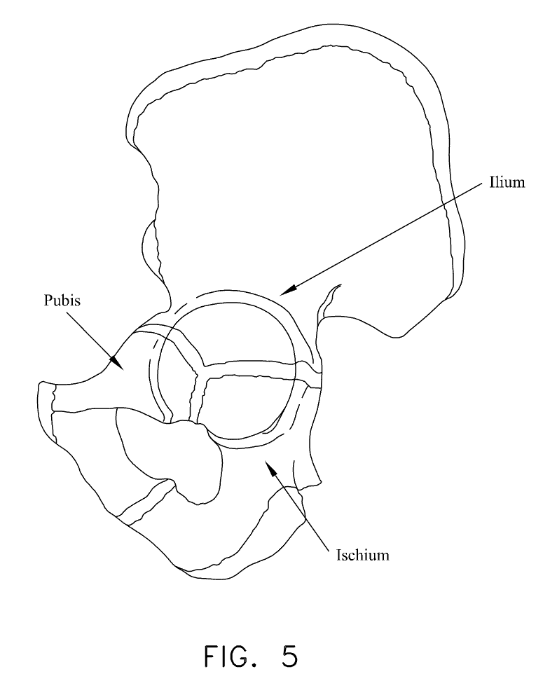

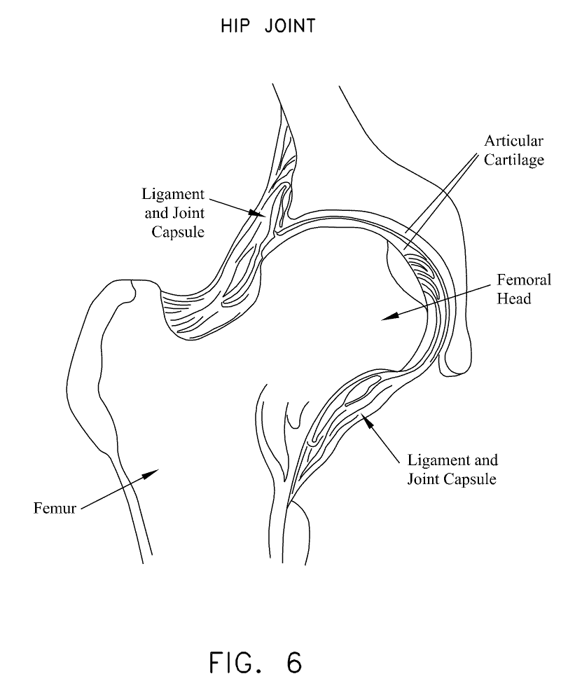

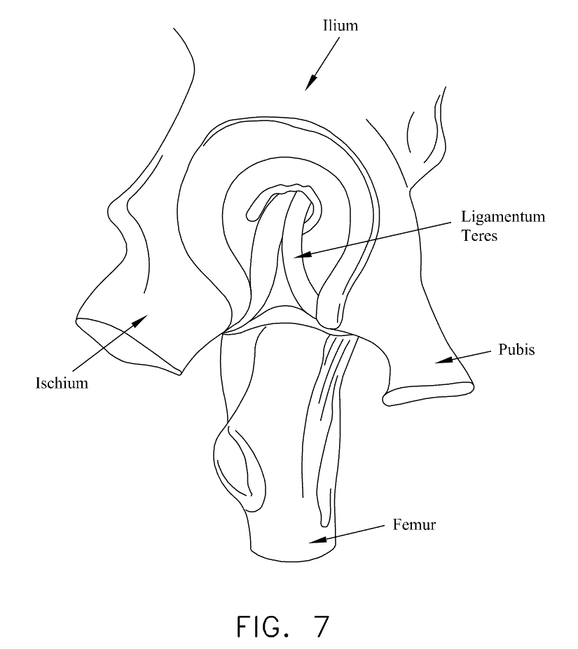

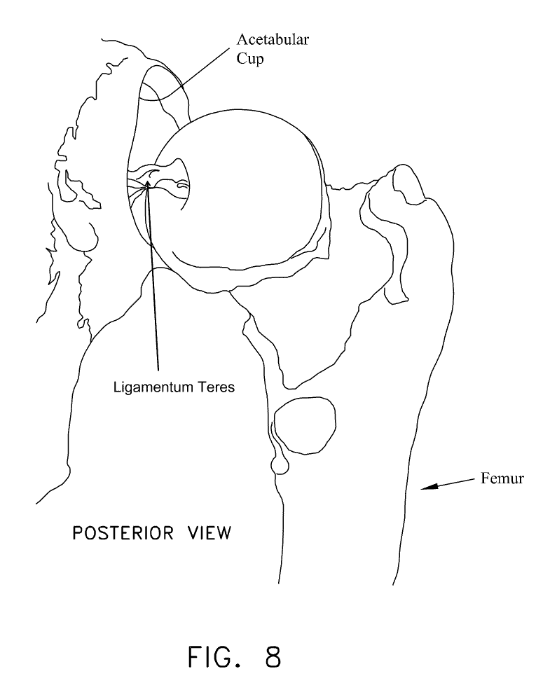

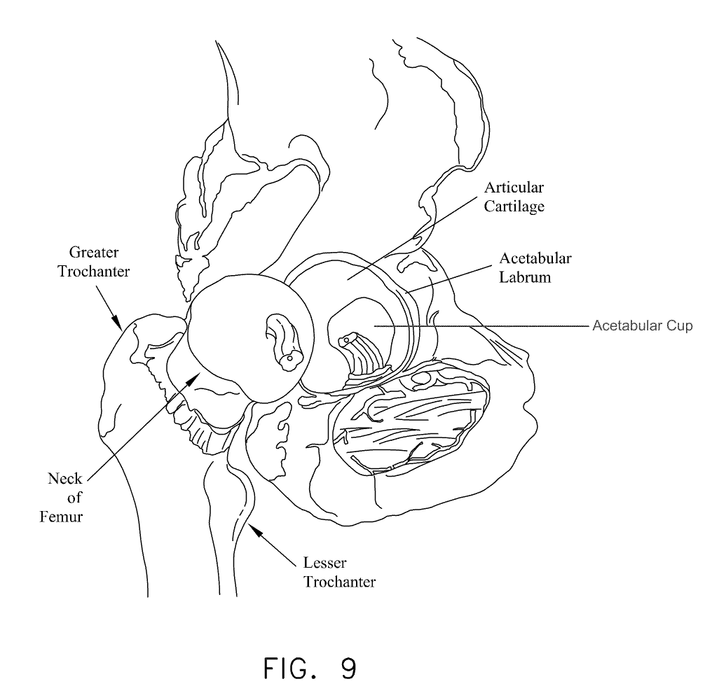

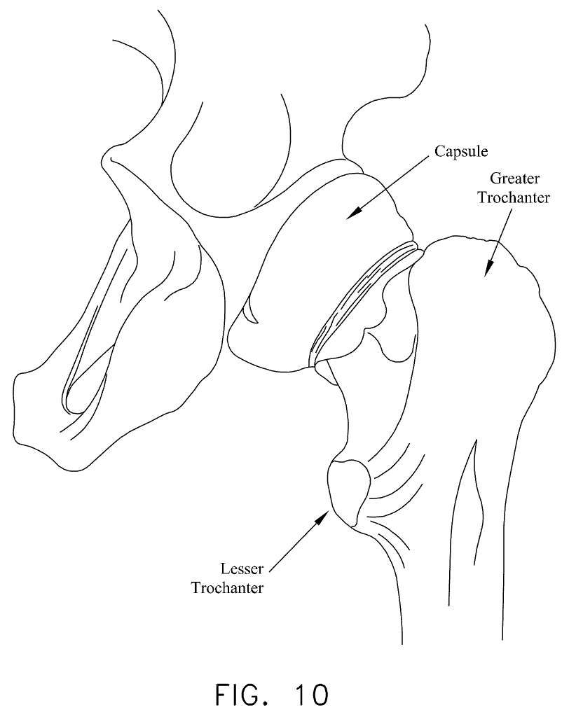

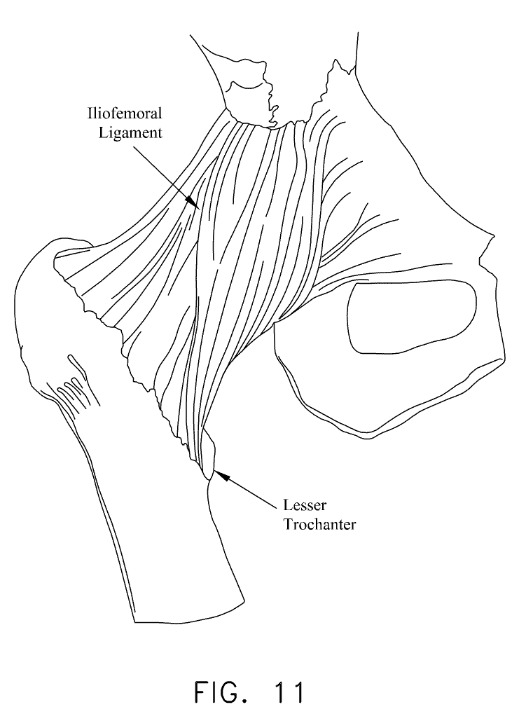

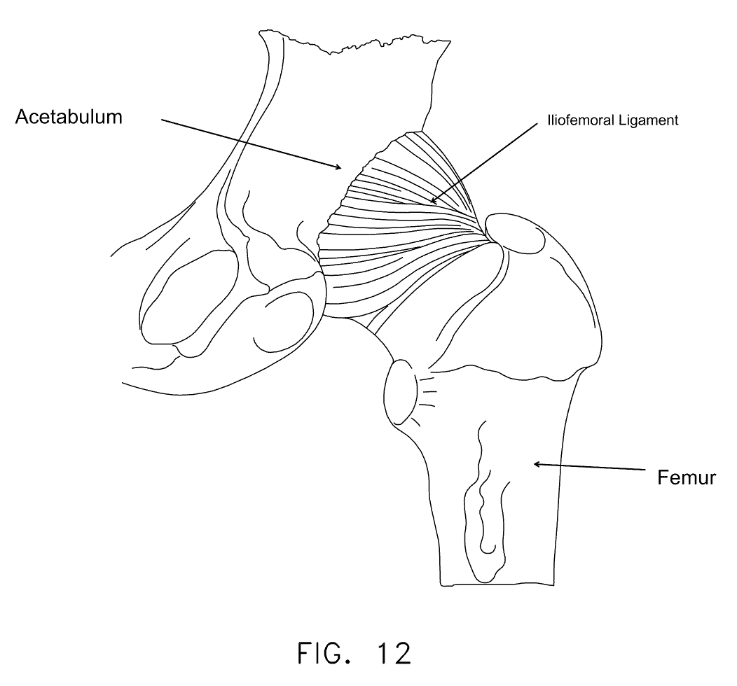

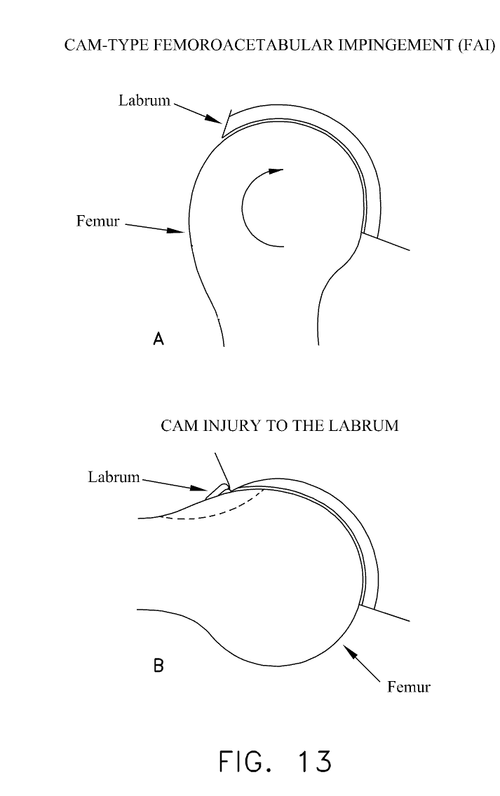

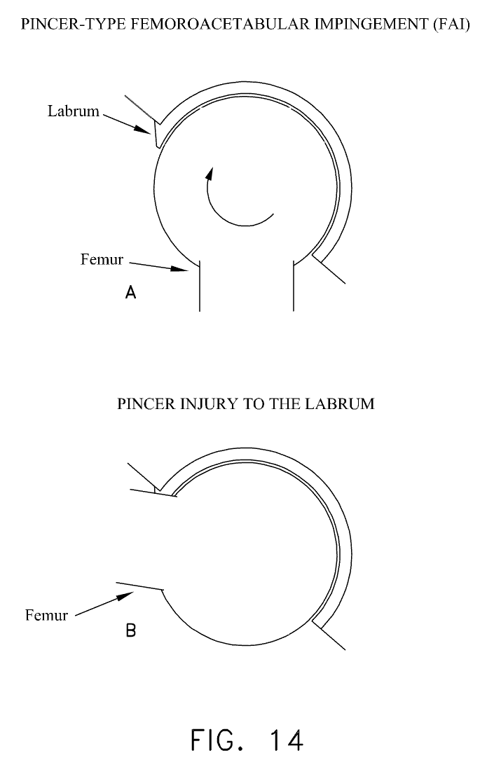

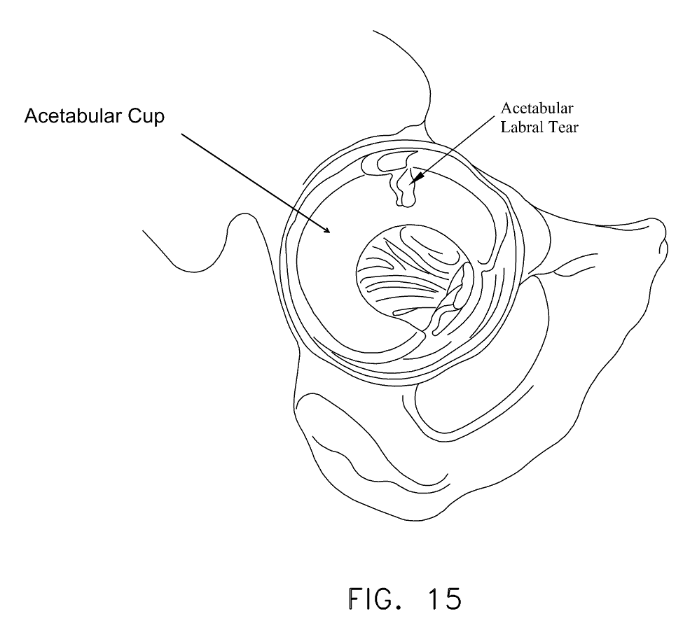

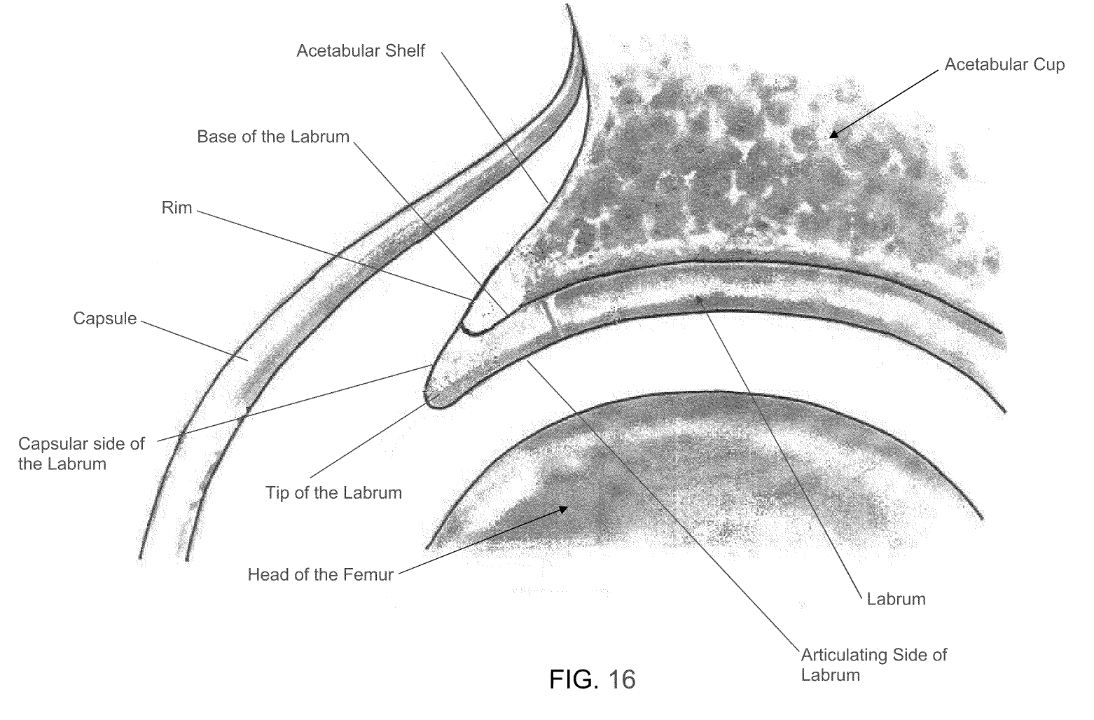

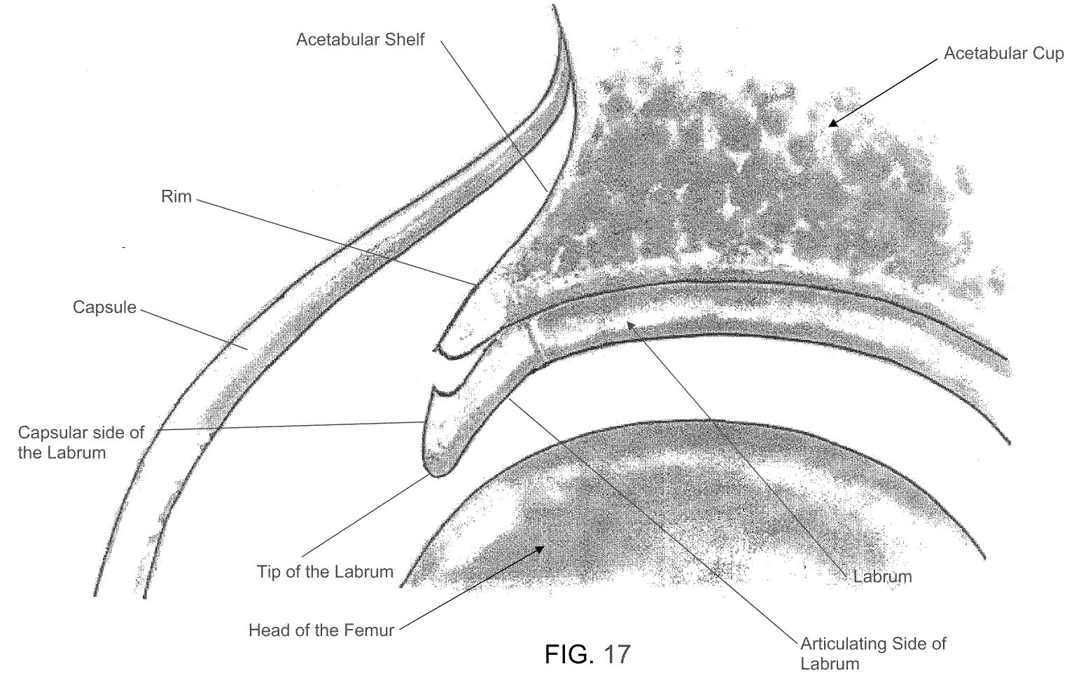

### DETAILED DESCRIPTION OF THE INVENTION

### Overview

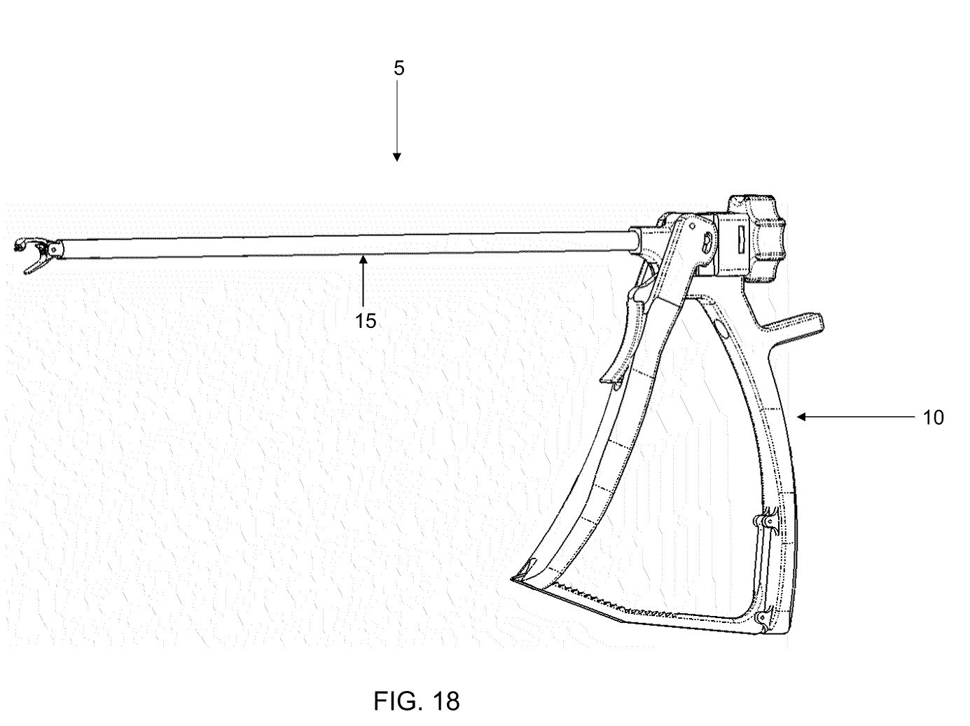

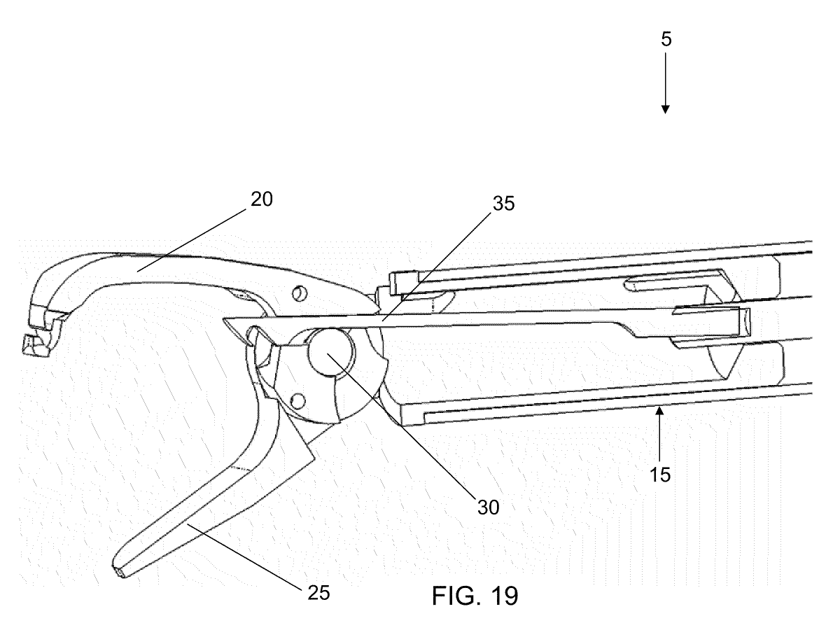

Looking next at FIGS. 18 and 19, there is shown a novel suture passer 5 formed in accordance with the present invention. Suture passer 5 generally comprises a handle 10 having a shaft 15 extending distally therefrom. A pair of articulating jaw members 20, 25 is pivotally mounted to the distal end of shaft 15 via a pivot pin 30. Pivot pin 30 may also be replaced by a set screw or other equivalent mechanism if desired. As will hereinafter be discussed in further detail, suture passer 5 is configured so that (i) the pitch of jaw members 20, 25 can be selectively varied relative to the longitudinal axis of shaft 15 so as to properly address tissue; (ii) jaw members 20, 25 can be selectively opened and closed relative to one another so as to grasp tissue therebetween; and (iii) a needle 35 can be selectively advanced and retracted relative to jaw members 20, 25 so as to pass suture through tissue grasped by the jaws.

Additionally, and as will hereinafter be discussed in further detail, various geometries may be provided for one or both of the inner faces of the jaw members so as to selectively configure the labrum grasped between the jaw members, and/or various geometries may be provided for the needle, whereby to influence the suture path through the labrum.

Jaw members 20, 25 are preferentially made of a stainless steel for rigidity, durability and precision. The jaw members may also be formed out of alternative metals such as titanium or Nitinol to take advantage of lower weight, increased flexibility or other material properties. Polymers may also be used to make the jaw members lower in weight, easier to manufacture and non-electrically conductive.

### The Pitch of Jaw Members 20, 25

As noted above, the pitch of jaw members 20, 25 can be selectively varied relative to the longitudinal axis of shaft 15 so as to allow suture passer 5 to properly address tissue.

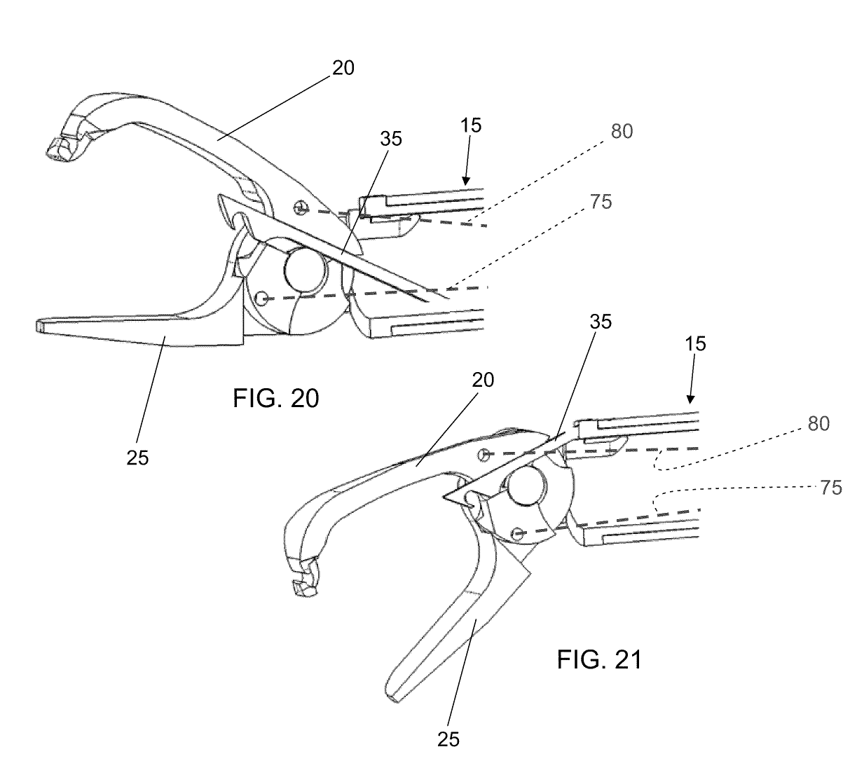

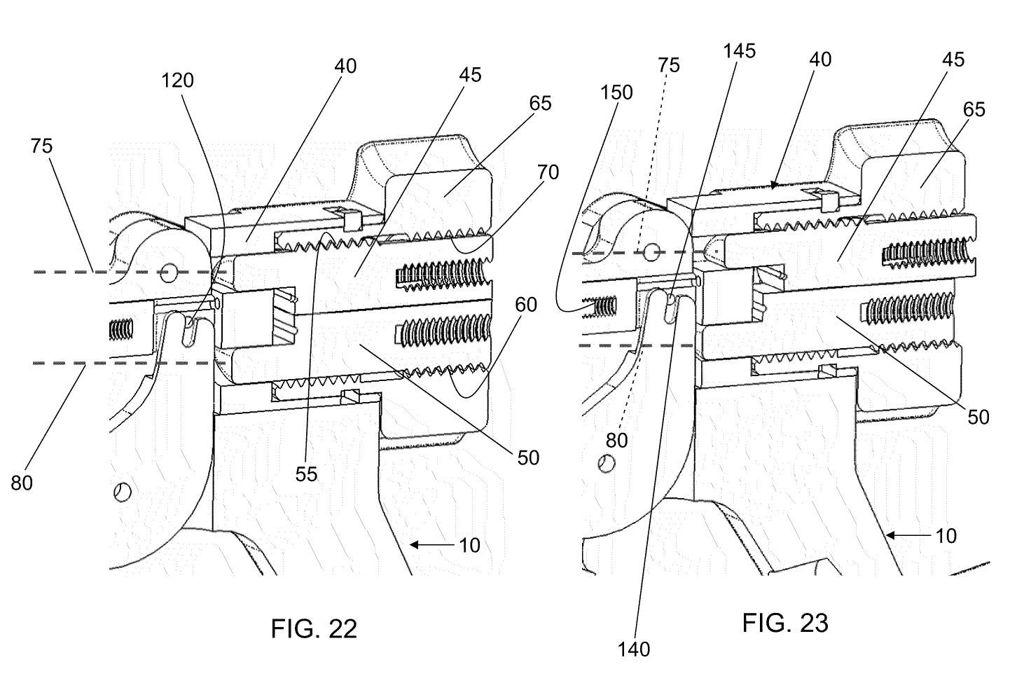

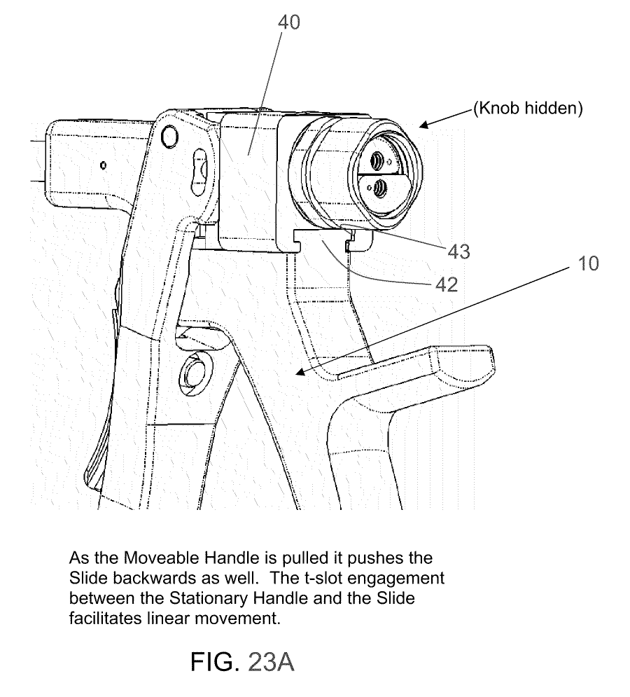

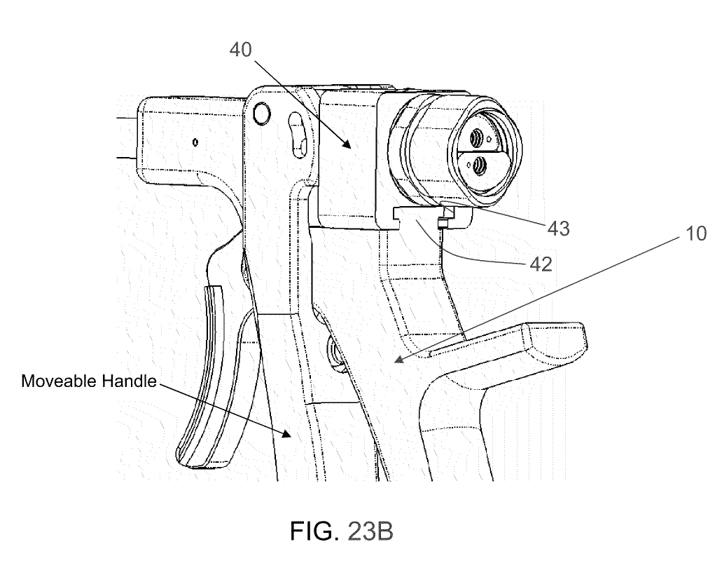

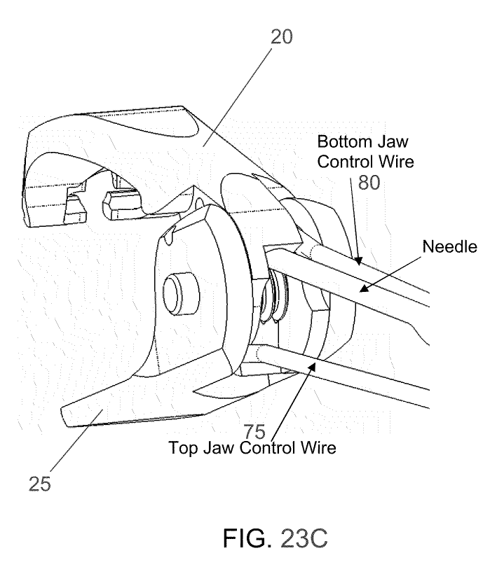

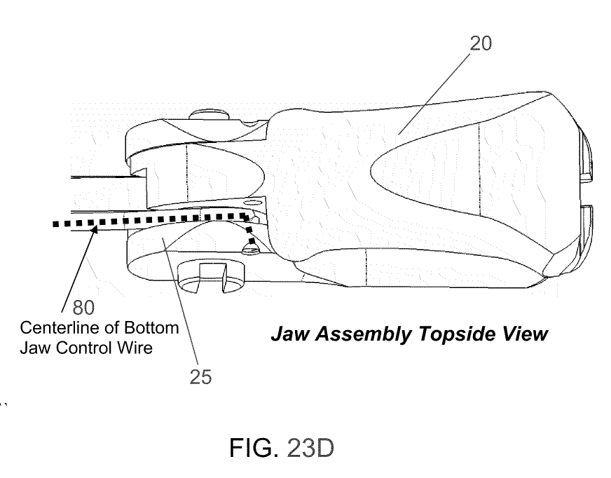

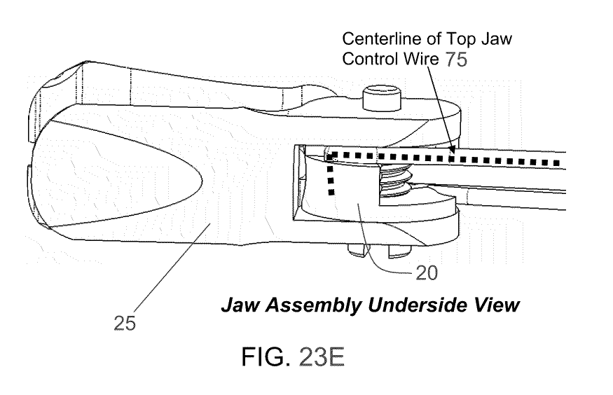

More particularly, and looking next at FIGS. 20-23, 23A, 23B, 23C, 23D and 23E, a slide 40 is slidably mounted to handle 10, e.g., via a tongue (42) and groove (43) construction. A pair of yokes 45, 50 are movably disposed within slide 40. Yokes 45, 50 comprise screw threads 55, 60, respectively. Screw threads 55, 60 are opposite turn threads, i.e., one is a clockwise turn thread and the other is a counter-clockwise turn thread. A knob 65 is rotatably mounted on yokes 45, 50. More particularly, knob 65 comprises an internal thread 70 which engages the aforementioned screw threads 55, 60 of yokes 45, 50. By virtue of this construction, rotation of knob 65 is one direction causes yoke 45 to retract proximally and yoke 50 to advance distally (FIGS. 22 and 23), and rotation of knob 65 in the opposite direction causes yoke 45 to advance distally and yoke 50 to retract proximally.

A first control rod 75 extends between jaw member 20 and yoke 45, and a second control rod 80 extends between jaw member 25 and yoke 50.

By virtue of the foregoing construction, when knob 65 is turned in a first direction, first control rod 75 is moved proximally and second control rod 80 is moved distally, such that jaws 20, 25 are pitched in an upward direction vis-à-vis handle 10 (FIG. 20). Correspondingly, when knob 65 is turned in the opposite direction, first control rod 75 is moved distally and second control rod 80 is moved proximally, such that jaws 20, 25 can be pitched in a downward direction vis-à-vis handle 10 (FIG. 21).

### Opening and Closing Jaw Members 20, 25

As noted above, jaw members 20, 25 can be selectively opened and closed relative to one another so as to grasp tissue therebetween.

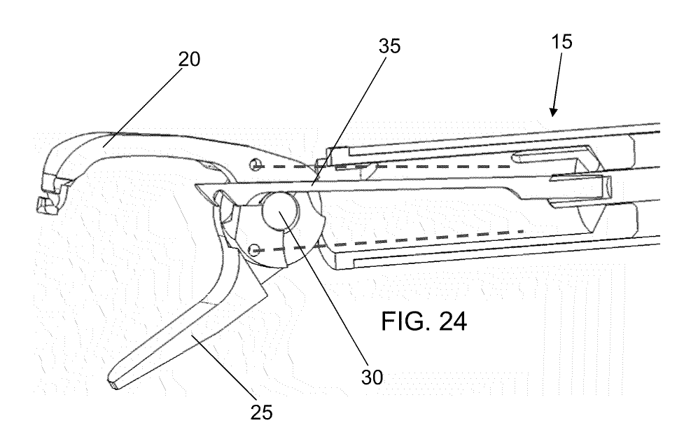

More particularly, and looking next at FIGS. 24-27, suture passer 5 comprises a lever arm 85 for opening and closing jaw members 20, 25. More specifically, lever arm 85 is pivotally mounted to handle 10 via a pivot pin 90. A spring 95 yieldably biases lever arm 85 away from handle 10.

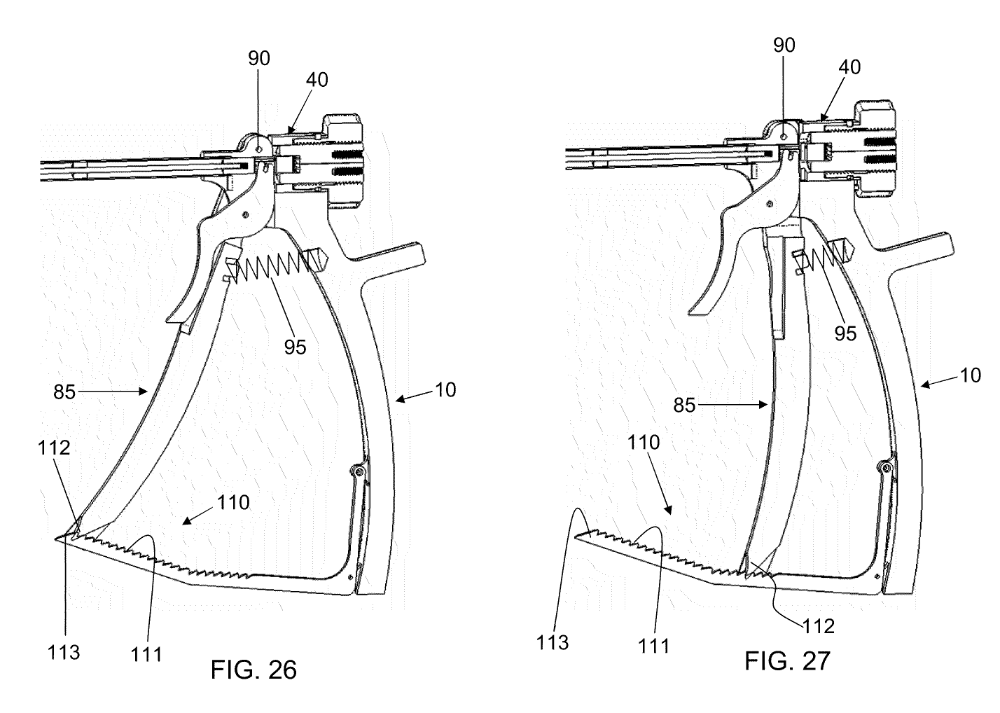

The configuration of the upper end of lever arm 85 is coordinated with the configuration of the distal end of slide 40 such that (i) when lever arm 85 is spring biased away from handle 10 in the position shown in FIG. 26, slide 40 is in its distalmost position, and (ii) when lever arm 85 is manually moved towards handle 10, slide 40 is moved proximally. In this respect, it should also be appreciated that yokes 45, 50 are carried on slide 40 and connected to jaws 20, 25, respectively, via control rods 75, 80, respectively.

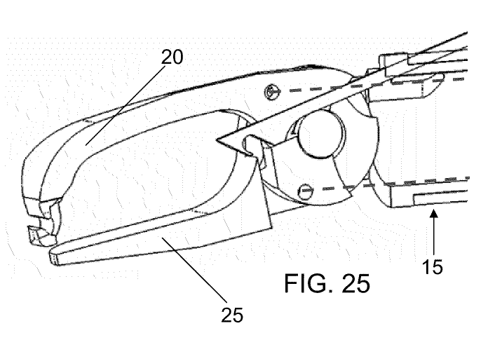

By virtue of the foregoing construction, when lever arm 85 is in its released position (i.e., the condition of FIG. 26), slide 40 is in its distalmost condition, and control rods 75, 80 position jaw members 20, 25 in their open position (FIG. 24). When lever arm 85 is pulled proximally towards handle 10 (i.e., the condition of FIG. 27), control rods 75, 80 position jaw members 20, 25 in their closed position (FIG. 25).

Significantly, since lever arm 85 is configured to act upon slide 40, and since yokes 45, 50 can assume variable positions on slide 40 according to the disposition of knob 65, lever arm 85 can open and close jaw members 20, 25 regardless of the disposition of yokes 45, 50 on slide 40, and hence regardless of the pitch of the jaw members relative to the shaft. Stated another way, due to the construction of suture passer 5, a single set of control rods 75, 80 can be used to control both the pitch of the jaws (via knob 65) and the opening/closing of the jaws (via lever arm 85), and these actions can be effected independently of one another.

In some circumstances it may be desirable to maintain jaw members 20, 25 in their closed position (FIGS. 25, 27) without requiring the continued application of manual pressure on lever arm 85. By way of example but not limitation, it can be helpful to maintain jaw members 20, 25 in their closed position while advancing suture passer 5 down a cannula to an internal surgical site, or while jaw members 20, 25 are grasping tissue (such as during tissue repositioning, suture passing, etc.). In any case, in order to maintain jaw members 20, 25 in their closed position, a ratcheting mechanism 110 can be provided on the outboard ends of handle 10 and lever arm 85 so as to releasably maintain lever arm 85 in a retracted position (FIG. 27). More particularly, handle 10 can include a plurality of teeth 111 configured to be engaged by a finger 112 formed on the outboard end of lever arm 85. Preferably, teeth 111 are inclined proximally so as to facilitate one-way motion of lever arm 85 toward to handle 10. When lever arm 85 is to be released, the distal tip 113 of ratcheting mechanism 110 can be pressed away from shaft 15, so as to free finger 112 from teeth 111. Thus it will be seen that ratchet mechanism 110 is rigid enough to provide a holding force for keeping the handles together, but be flexible enough to allow advancement to the next ratchet pawl.

### Needle 35 for Suture Passing

As noted above, needle 35 can be selectively advanced and retracted relative to jaw members 20, 25 so as to pass suture through tissue.

More particularly, and looking next at FIGS. 22, 23 and 28-32, suture passer 5 comprises a transverse shaft 115 connected to needle 35 at one end and to a carriage 120 (FIG. 22) at the other end. A trigger 125 is pivotally mounted to handle 10 via a pivot pin 130. Trigger 125 comprises a slot 140 which receives a pin 145 extending outboard of carriage 120. A spring 150 (FIG. 23) biases trigger carriage 125 proximally.

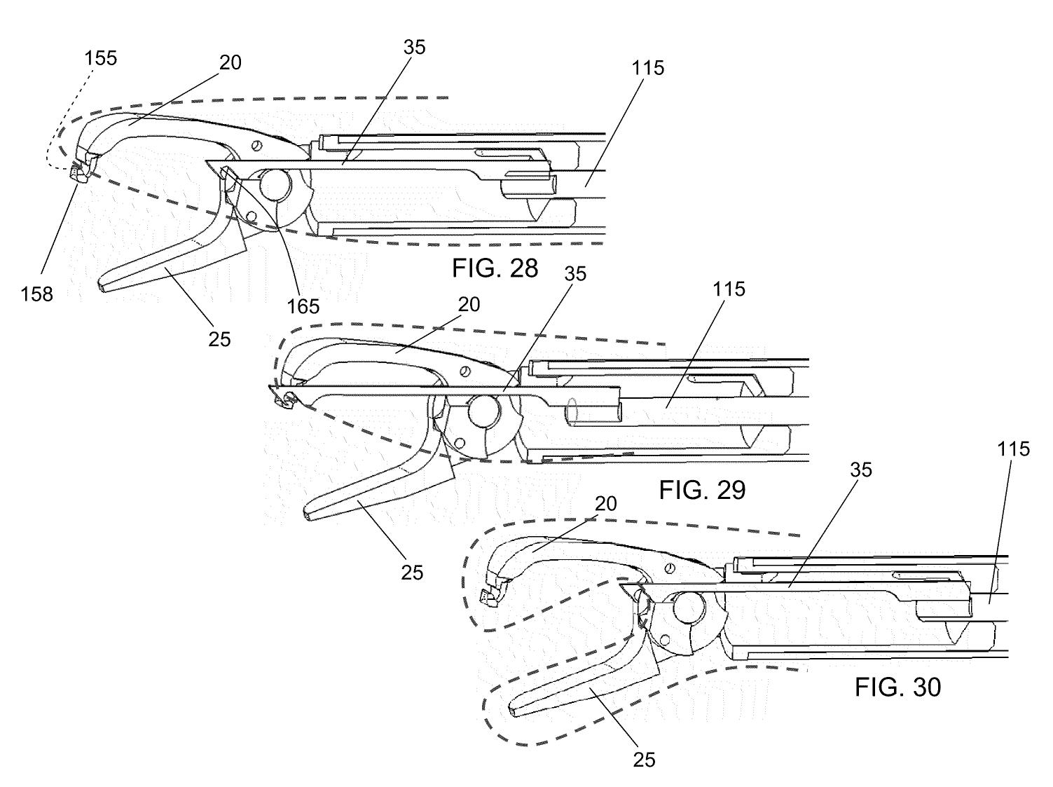

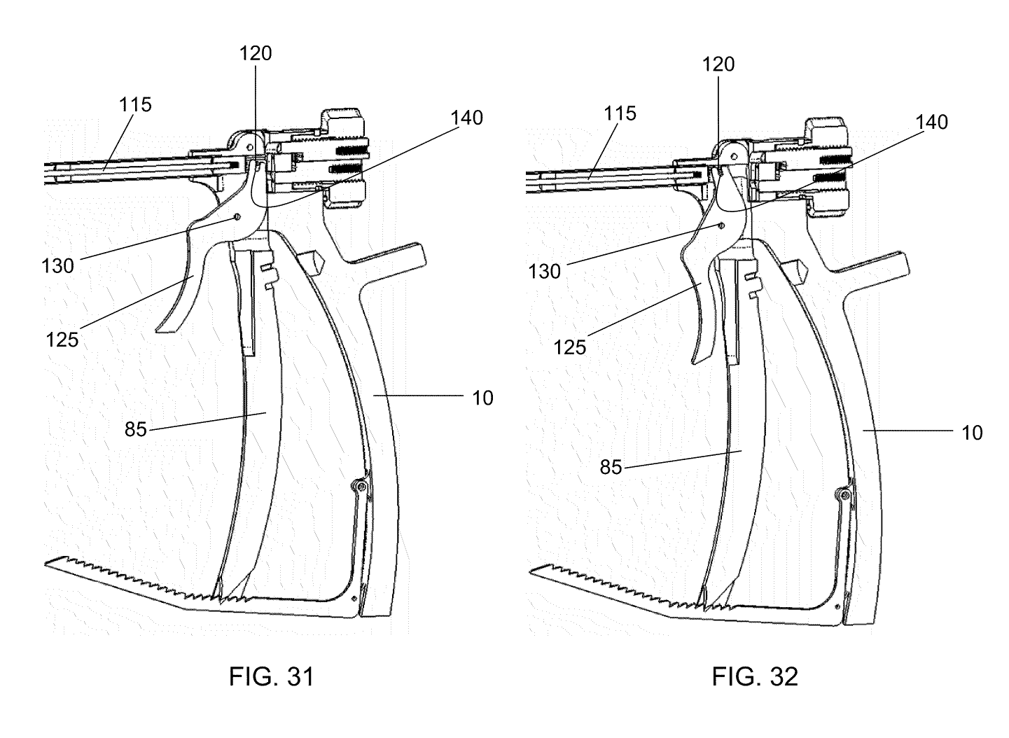

By virtue of the foregoing construction, when trigger 125 is in its released condition, i.e., the condition of FIG. 31, carriage 120 is in its proximal position, so that needle 35 is in its retracted position (FIG. 28). When trigger 125 is pulled proximally, carriage 120 is moved distally, so that needle 35 is in its projected position (FIG. 29).

Significantly, trigger 125 and lever arm 85 are configured so that trigger 125 may not be pulled if lever arm 85 is in its distal position (i.e., FIG. 26), and trigger 125 may only be pulled if lever arm 85 is in its proximal position (i.e., FIG. 31). As a result, needle 35 can only be advanced (i.e., by pulling trigger 125) when lever arm 85 is in its distal position, i.e., when jaw members 20, 25 are in their closed position.

When it is desirable to retract the needle from its advanced position, trigger 125 is released, allowing spring 150 to return carriage 120 to its proximal position, whereby to also return needle 30 to its retracted position (FIG. 30).

It should be appreciated that when needle 35 is advanced to its distal position, it projects through an opening 155 formed in jaw member 20 so as to retrieve a suture held thereon, as will hereinafter be discussed in further detail. More particularly, jaw 20 comprises a suture support 158 adjacent to opening 155, and a groove 165 formed adjacent to the sharp distal tip of needle 35, so that a suture 167 supported in suture support 158 can be picked up by groove 165 of needle 35, in a “crochet hook” manner, whereby to draw the suture through tissue, as will hereinafter be discussed in further detail. The channel of suture support 158 is preferentially sized to hold a polyethylene braided suture of United States Pharmacopeia size #2. Alternatively, the channel of suture support 158 could be sized to hold any different size of suture, any different material of suture, or other construction of suture as may be beneficial for improving the use and outcome of the suture passer.

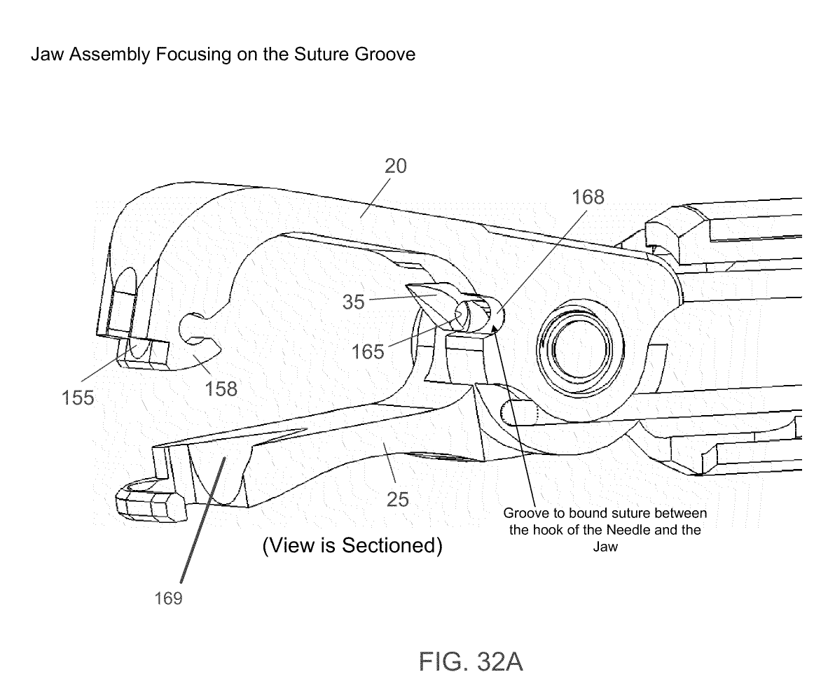

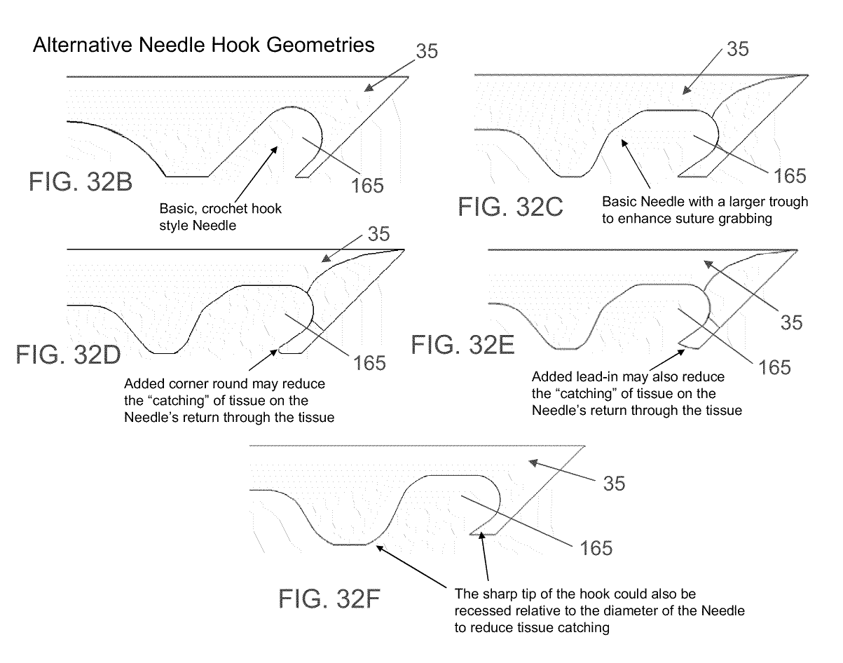

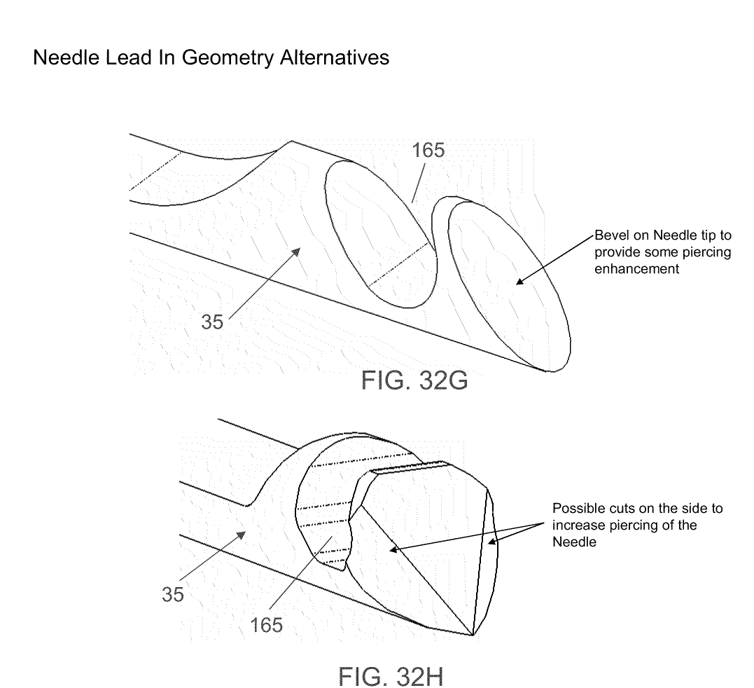

Preferably, and looking now at FIG. 32A, upper jaw member 20 includes a groove 168 for receiving suture 167 when needle 35 is retracted, such that suture 167 can be securely captured between groove 165 of needle 35 and groove 168 of upper jaw member 20. Among other things, this feature can be helpful when suture passer 5 is removed from the surgical site, since it lessens the chance that the suture will become disengaged from the suture passer when the suture passer is withdrawn out of the body.

It will be appreciated that, since jaw members 20, 25 are designed to assume various pitches relative to the longitudinal axis of shaft 15, needle 35 is preferably configured to bend along its length, so that the needle can pass out of shaft 15 and into jaw members 20, 25 when the jaw members are disposed in a variety of different head pitches. To this end, the needle is preferentially made of a flexible metal material such as Nitinol. It may also be useful to have a more rigid material that could improve piercing and strength of the device, such as stainless steel. Yet another alternative is to use a combination of materials to combine the rigidity and strength of stainless steel and the flexibility of Nitinol. Coating materials may also be used to improve the hardness of the surface of the needle, lubricity or visibility of the needle. Furthermore, needle 35 may be thinned along its length so as to facilitate bending, and/or the needle may be otherwise pre-formed so as to allow the needle to flex and thereby pass around corners as necessary.

In addition to the foregoing, jaw members 20, 25 and needle 35 are preferably configured so that the “crochet hook” portion of the needle makes a positive interference with the suture, with the needle flexing out of the way upon engagement with the suture and thereafter coming back “down” on suture so as to capture the suture within needle groove 165. In other words, the needle bends as necessary so as to accommodate the suture position. Thus, with this device, the suture and needle are designed to occupy the same space, such that when the needle is advanced, either the needle or the suture must deviate from the commonly shared space and then, after deviation, return to its previous position, with the suture captured then in the groove of the needle—and in the preferred form of the invention, the needle is configured to flex away from the intersection point. Alternatively, features may be provided on jaw members 20, 25 (such as a transverse trough on the top jaw member) which could have other features (e.g., springs, thin sections, etc.) that permit the suture to temporarily move away from the needle, instead of the needle moving out of the way of the suture as discussed above.

It should also be appreciated that needle 35 can have various hook geometries. See, for example, FIGS. 32B-32H. Among other things, needle 35 can have a reverse angle portion for catching/hooking suture. And needle 35 can include a corner round on the tip of the hook to reduce the drag of the hook when pulled back through the tissue. And the leading portion of the needle can be chamfered in both the lateral aspect as well as the longitudinal aspect. This lateral chamfering can help the needle ride over the suture, or push the suture out of the way, when the needle engages the suture. The longitudinal chamfering can help form a sharp, central tip for the needle, thereby facilitating precise and easy piercing of the tissue. However, it should also be appreciated that the needle can also be blunt, though this construction requires higher insertion forces to pass the needle through the tissue.

In FIGS. 28-30, suture support 158 is shown holding suture 167 on the distal side of opening 155. However, and looking now at FIG. 32A, it should also be appreciated that suture support 158 may be configured to hold the suture on the proximal side of opening 155. In this respect it should be appreciated that different constructions can be beneficial for different purposes. More particularly, distal suture loading may make it easier to load the suture onto the device. Proximal suture loading may make it easier to remove the suture from the device.

### Operation

Suture passer 5 can be used to pass suture through tissue for a variety of purposes.

By way of example but not limitation, suture passer 5 can be used to pass suture through a portion of the labrum which has previously been detached from the acetabulum, either deliberately (e.g., as part of a procedure to address pincer-type impingement) or accidentally (e.g., through accident or injury).

For the purposes of illustrating the operation of suture passer 5, operation of suture passer 5 will now be discussed in the context of its use to re-attach the labrum during a procedure to address pincer-type impingement.

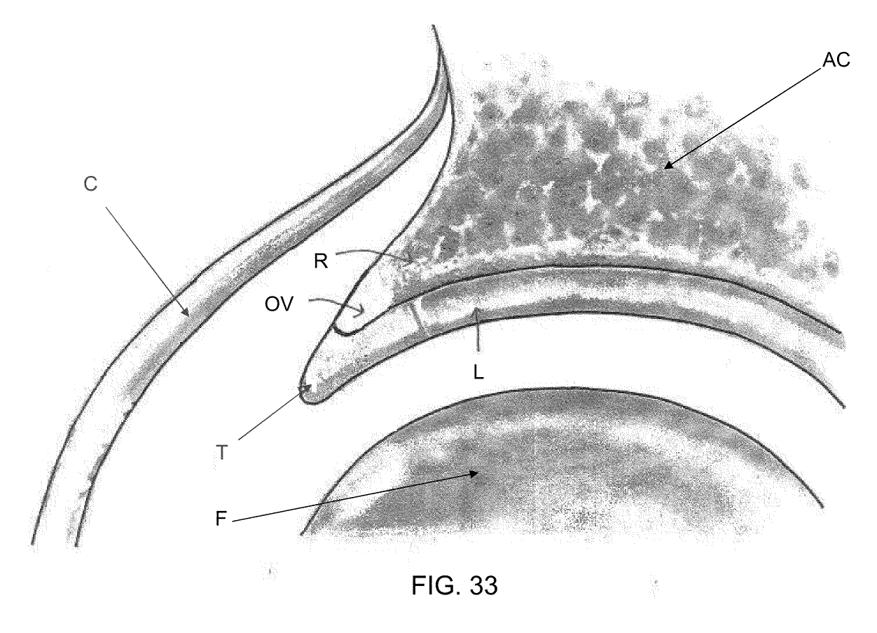

More particularly, and looking now at FIG. 33, overgrowth OV at the rim R of the acetabular cup AC can result in impingement of femur F on overgrowth OV when the hip moves through its normal cycle. This impingement can cause discomfort for the patient and, over time, can ultimately result in deterioration of the hip joint. Among other things, such impingement can frequently result in damage to the labrum L, particularly in the region of tip T of labrum L.

For this reason, it is often desirable to remove overgrowth OV via a debridement procedure. Of course, in order to spare the labrum during this debridement procedure, it is first necessary to release the labrum from overgrowth OV and then, after the overgrowth has been removed, re-attach the labrum to the acetabulum. Such re-attachment of the labrum to the acetabulum is typically accomplished by deploying a suture anchor in the acetabulm so that one or more strands of suture extend from the acetabulum, and then passing the suture through the labrum so that the suture can support the labrum against the acetabulum.

However, as noted above, this procedure is technically challenging, with passage of the suture through the labrum being one difficult aspect of the procedure. This is particularly true inasmuch as the suture should be passed through the labrum so that the suture does not open on the articular side of the labrum (i.e., so that the suture does not open on the side of the labrum facing femur F), and the suture should be passed through the labrum so that the labrum is re-attached to the acetabulum with anatomically-correct positioning.

Suture passer 5 can be used to facilitate passing the suture through the labrum so as to simplify proper re-attachment of the labrum to the acetabulum. Among other things, and as will hereinafter be discussed in further detail, suture passer 5 can be used to pass the suture through the labrum so that the suture does not open on the articular side of the labrum, and so that the suture is passed through the labrum so that the labrum is re-attached to the acetabulum with anatomically-correct positioning.

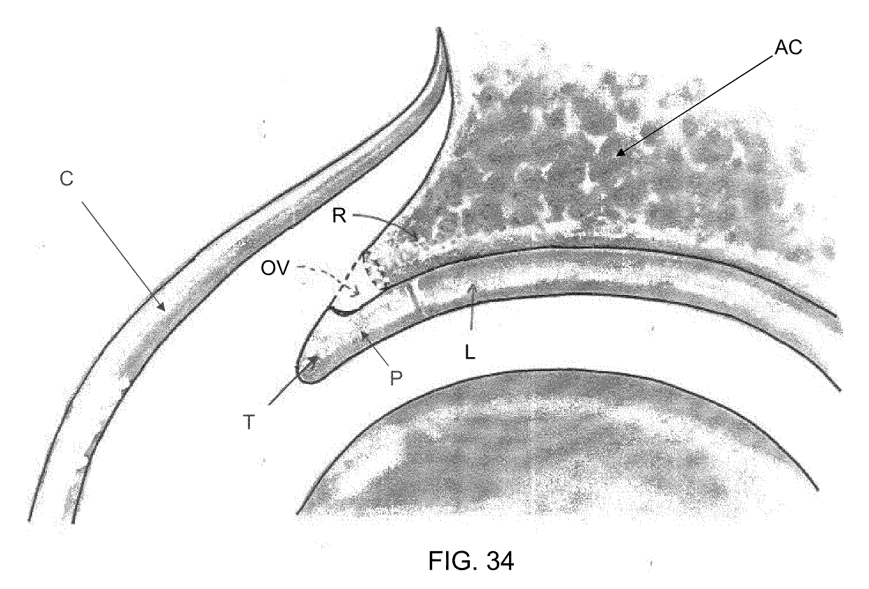

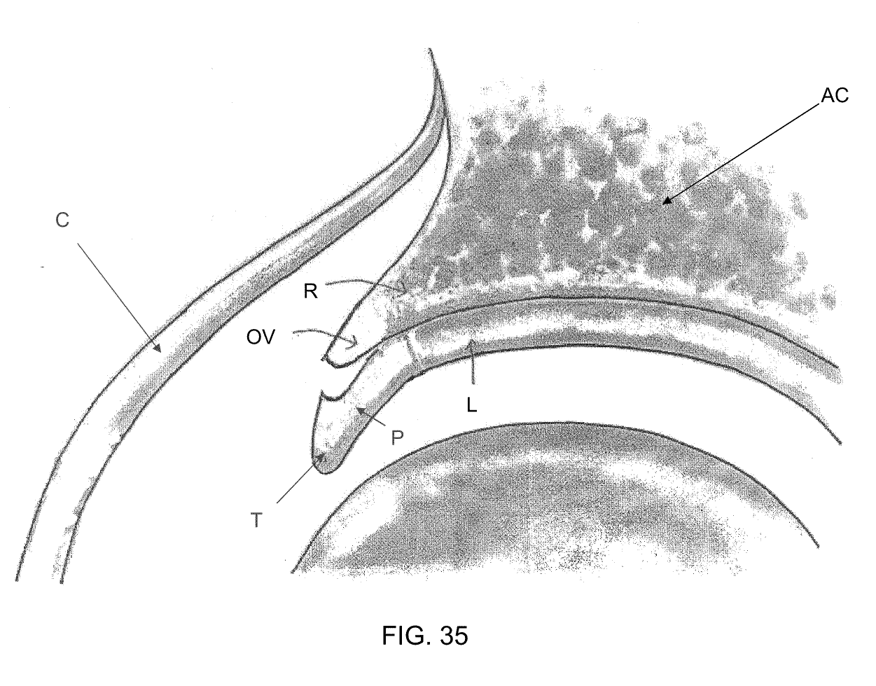

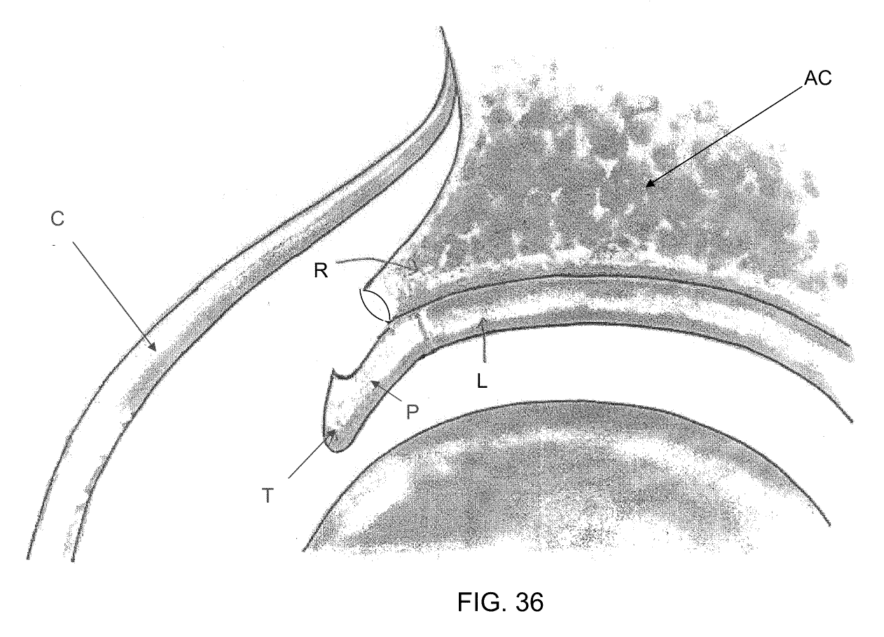

More particularly, during the debridement procedure, the surgeon first identifies the overgrowth OV which is to be removed (FIG. 34). Then, in order to spare the labrum, the surgeon carefully detaches the portion P of labrum L which overlies the overgrowth OV which is to be removed (FIG. 35). Once portion P of labrum L has been detached from the acetabulum, overgrowth OV can be removed, e.g., by debridement (FIG. 36).

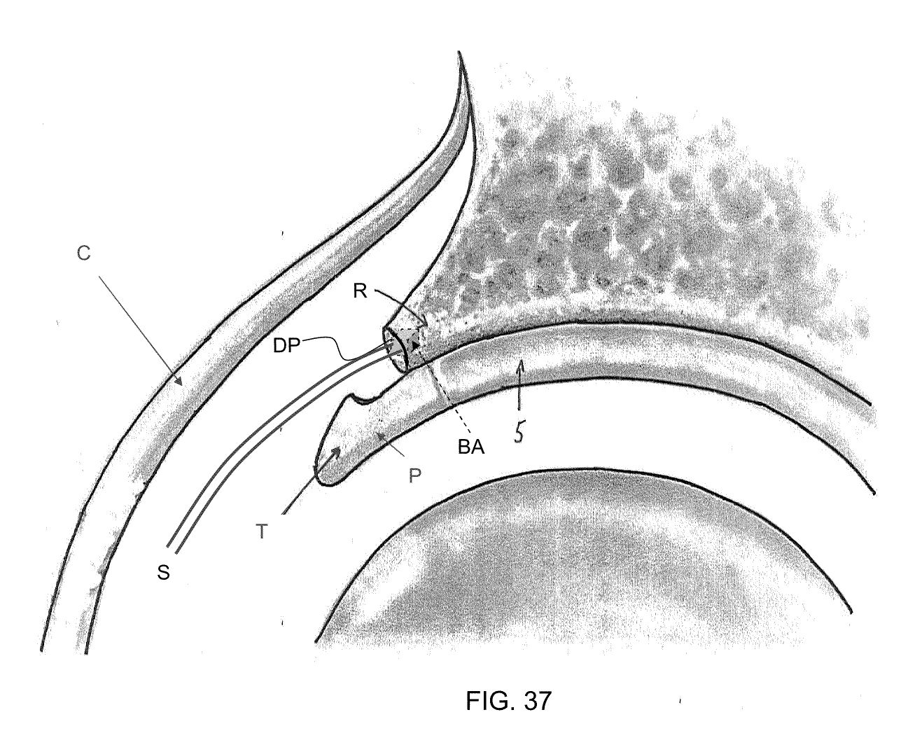

After overgrowth OV has been removed, it is necessary to re-attach portion P of labrum L to the acetabulum (FIG. 36). This can be done by deploying a bone anchor BA (FIG. 37) in the debrided portion DP of the acetabulum so that suture strands S extend out of debrided portion DP. Then suture passer 5 can be used to pass one or more of suture strands S through labrum portion P so that labrum portion P can be re-attached to debrided portion DP. In accordance with the present invention, and as will hereinafter be discussed in further detail below, suture passer 5 is used to pass suture strands S through labrum portion P so that the suture strands are substantially aligned with tip T of labrum L, and/or enter or exit labrum portion P on the side of the labrum facing capsule C, i.e., on the capsular side of the labrum. Such suture placement helps ensure that the suture does not open on the articular side of labrum L, which could cause damage to the cartilage of the joint, and helps ensure that labrum L is re-attached to the acetabulum with anatomically-correct positioning.

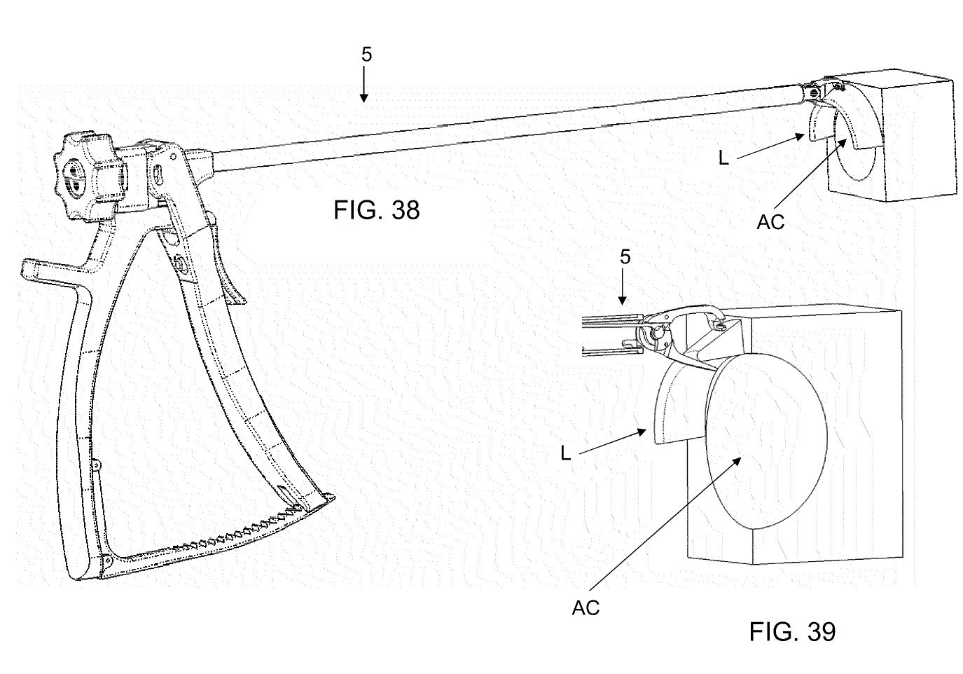

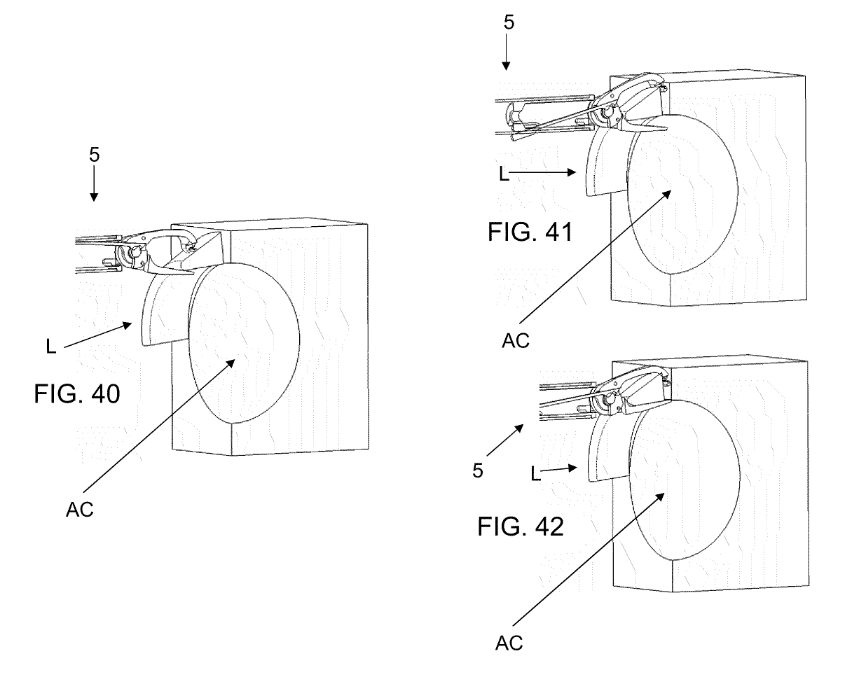

More particularly, and looking now at FIGS. 38-44, suture passer 5 is generally first configured so that its jaw members 20, 25 are in an open position, with jaw members 20, 25 in their “neutral” pitch position, and with needle 35 in its retracted position (FIG. 28). Then, while suture passer 5 is located outside the body, one of the suture strands S emanating from bone anchor BA is loaded into suture support 158. Then suture passer 5 has its jaw members 20, 25 placed into their closed position (FIG. 25) by moving lever arm 85 toward handle 10, and then the suture passer is advanced to the surgical site.

Suture passer 5 is advanced until it is positioned adjacent to detached portion P of labrum L. Then lever arm 85 is released so that jaw members 20, are opened. Next, suture passer 5 is positioned so that the labrum is disposed between open jaw members 20, 25 (FIG. 39). Depending upon the disposition of the labrum and the angle of suture passer approach, if desired, the pitch of jaw members 20, 25 may be adjusted as necessary so as to align the major plane of the jaw members with the major plane of the labrum. See FIGS. 38-41. Then lever arm 85 is pulled proximally so as to close jaw members 20, 25 onto the labrum (FIG. 42). The jaw members are closed so as to avoid damaging the labrum, while at the same time gripping the labrum firmly, so that the suture passer can position the labrum as anatomically appropriate and stabilize the labrum for later passage of needle 35 therethrough.

The surgeon can now use suture passer 5 to manipulate the labrum into a desired position. This may be done by appropriately manipulating handle 10 and/or by adjusting the pitch of jaw members 20, 25. In this respect it will be appreciated that, given the limited range of motion normally available to the surgeon when operating endoscopically within the hip, the ability to adjust the pitch of jaw members 20, 25 relative to shaft 15 after the labrum has been grasped by suture passer 5 provides the surgeon with significant additional ranges of motion. This facilitates proper positioning of the labrum relative to bone, thereby significantly enhancing proper anatomical positioning of the labrum during the repair procedure.

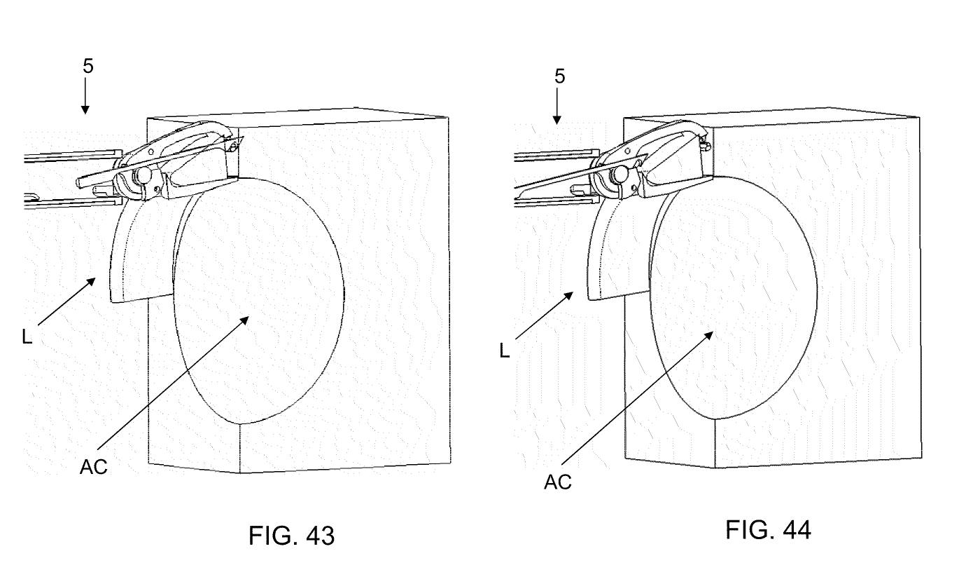

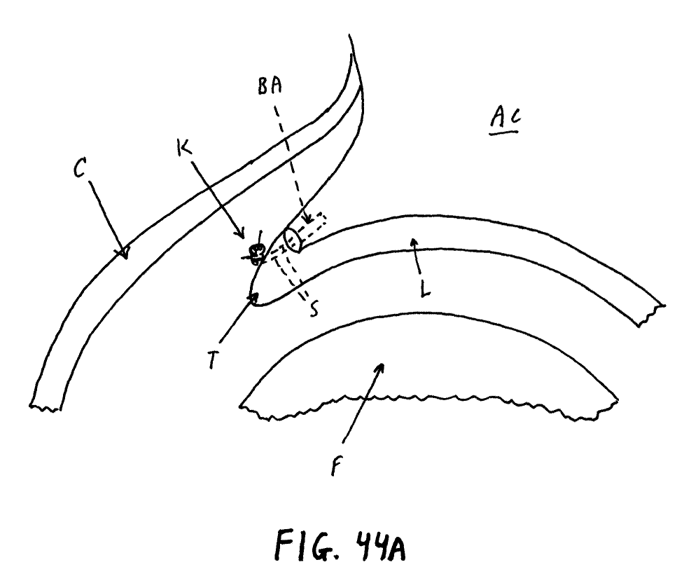

When the labrum is in the desired position, needle 35 can be advanced to, and through, the labrum by pulling trigger 125 (FIG. 43). It should be appreciated that as needle 35 is advanced to and through the labrum, needle 35 moves substantially parallel to the plane of tip T of labrum L, staying within the labrum during the entirety of the stroke and never opening on the articular face of the labrum. In one preferred form of the invention, needle 35 enters labrum L on the end surface of tip T. In another preferred form of the invention, needle 35 enters labrum L on the capsular side of the labrum. Needle 35 advances forward, through labrum L, until the needle projects through opening 155 formed in jaw member 20, such that suture S held in suture support 158 is engaged by groove 165 of needle 35. Trigger 125 is thereafter released, retracting needle 35 back through the labrum, carrying suture S with it (FIG. 44). As a result, suture S extends through the labrum, substantially aligned with the plane of tip T, exiting on the end surface of tip T or the capsular side of the labrum. This approach ensures that the suture does not open on the articular side of labrum L, which could cause damage to the cartilage of the joint, and helps ensure that labrum L is re-attached to the acetabulum with anatomically-correct positioning. Suture S can thereafter be tightened and knotted off, whereby to re-attach the labrum to the acetabulum. Again, inasmuch as suture S exits labrum L either on the capsular side of the labrum or at the tip T of the labrum, the knot will be located either on the capsular side of the labrum (preferably near the base of the labrum, substantially on top of the bone and anchor) or even at tip T, and it will not be positioned on the articular side of the labrum. Thus, there is no danger that the knot may engage and thereby damage the articular cartilage of the joint. See, for example, FIG. 44A, which shows labrum L being secured to the acetabulum via a bone anchor BA and sutures S, wherein the knot K lies on the capsular side of the labrum.

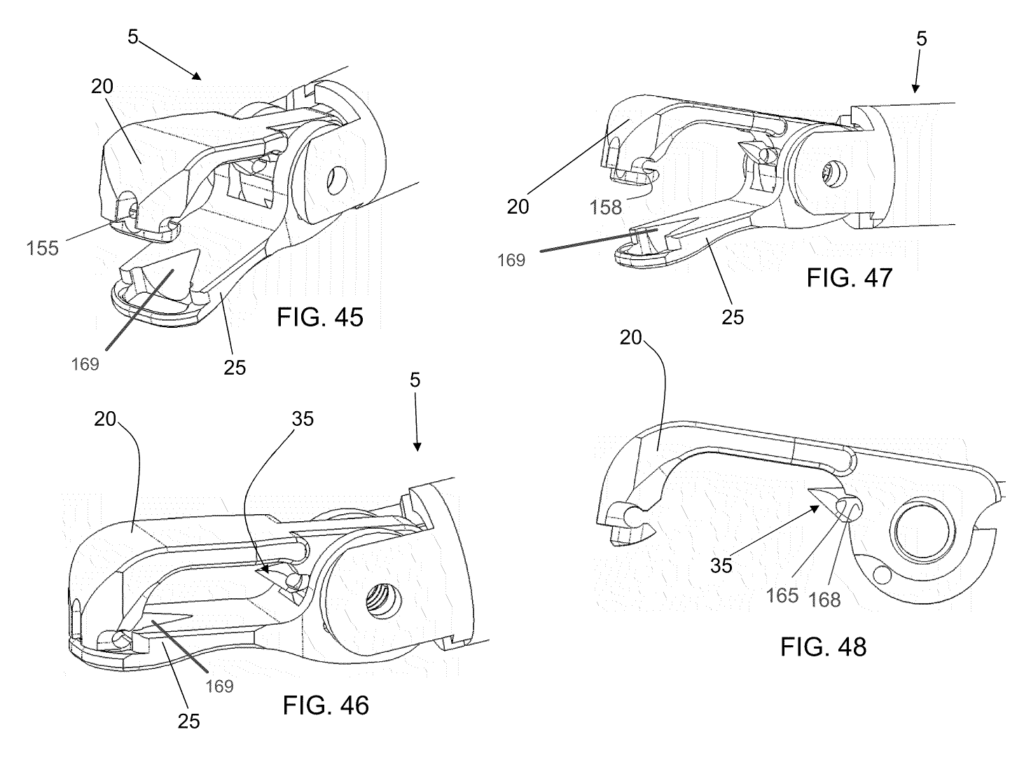

FIGS. 45-48 show one preferred structure for jaw members 20, 25. In FIGS. 45-48, upper jaw member 20 has its suture support 158 facing proximally. Lower jaw member 25 includes a distal groove 169 (also shown in FIG. 32) which permits needle 35 to be advanced through opening 155 in upper jaw member 20 even when jaw members 20, 25 are pitched downward relative to the longitudinal axis of shaft 15. FIG. 48 also illustrates how groove 165 of needle 35 cooperates with groove 168 of lower jaw member 25 so as to form a nest for suture 167 when needle 35 is retracted.

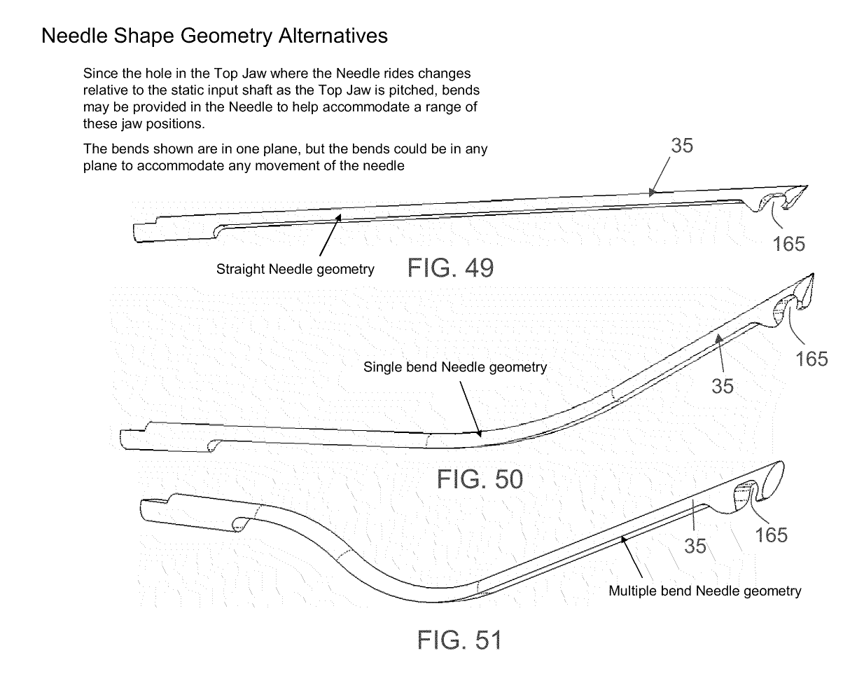

Looking next at FIGS. 49-51, it is also possible to provide needle 35 with various configurations which may enhance passing the suture through the labrum across a range of different jaw positions (i.e., pitches). More particularly, the needle may be formed substantially straight (FIG. 49), or the needle may be formed with a single bend (FIG. 50) or with multiple bends (FIG. 51)). Where the needle is formed with multiple bends, the bends may be in more than one plane.

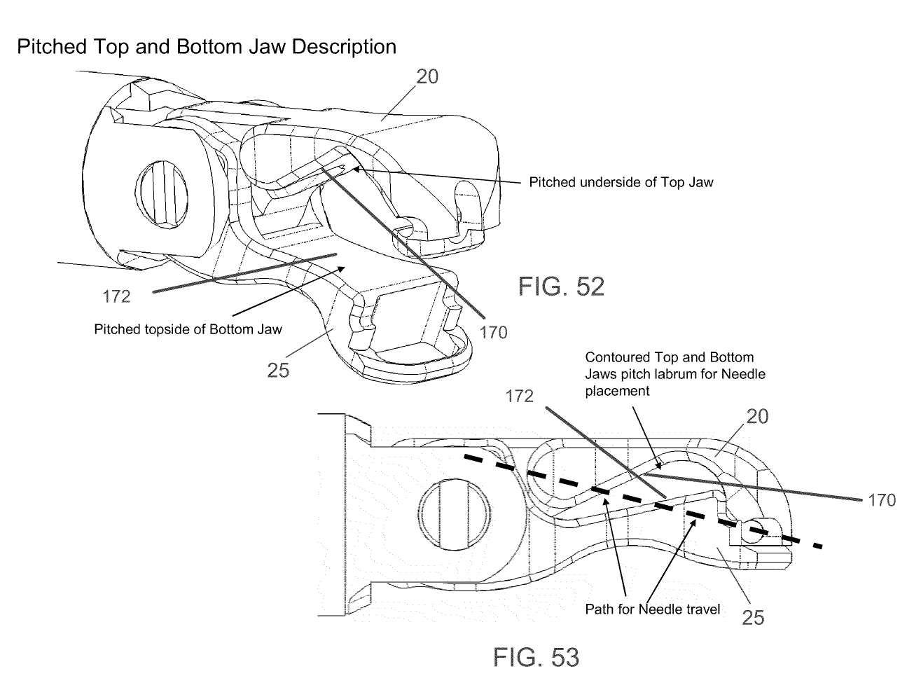

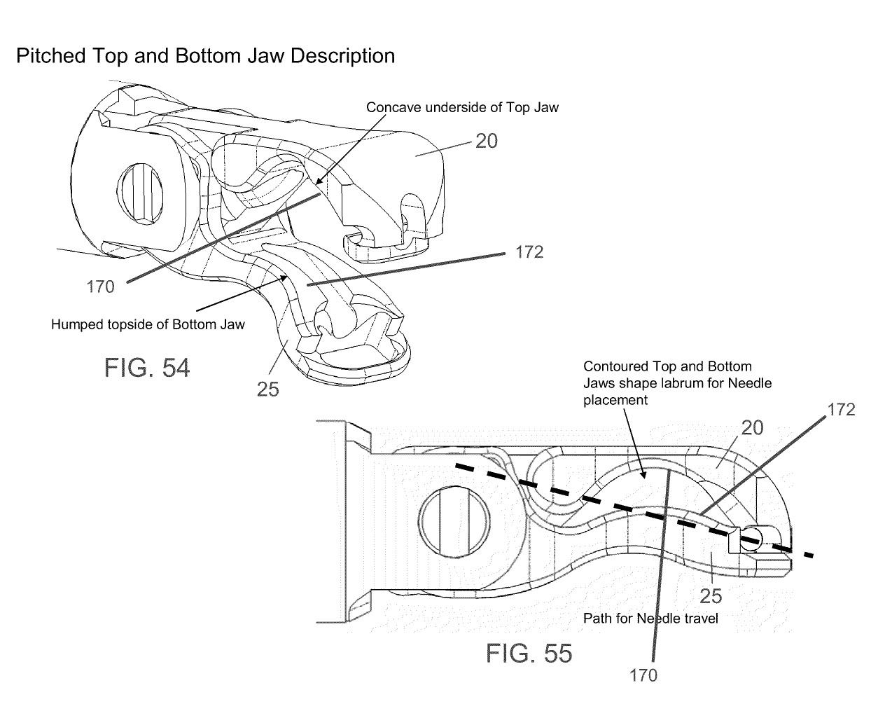

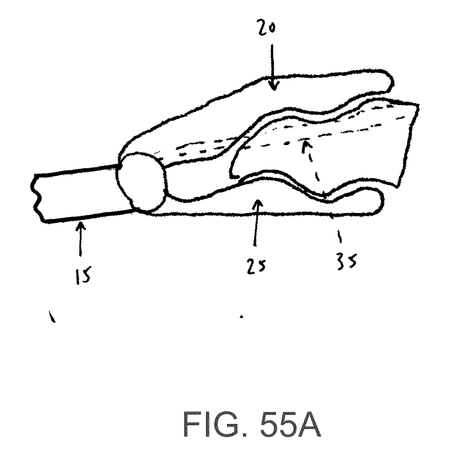

It is also possible to provide jaw members 20, 25 with labrum-engaging surfaces designed to contour the labrum therebetween. See, for example, FIGS. 52 and 53, which shown upper jaw member 20 with a pitched labrum-engaging surface 170 and lower jaw member 25 with a pitched labrum-engaging surface 172, such that when the labrum is gripped between the jaw members, the labrum assumes a non-planar configuration. See also FIGS. 54 and 55, which show curved labrum-engaging surfaces 170, 172. Providing jaw members 20, with labrum-contouring surfaces can be advantageous, since when the needle is thereafter passed through the labrum in a straight line and the labrum is subsequently released, the suture extends along a curved path. See, for example, FIG. 55A. This can be used to beneficially position the suture in more favorable positions so as to avoid contact with the articulating cartilage and to enhance the mechanical interaction of the labrum and suture so as to create a more anatomic reconstruction, for example.

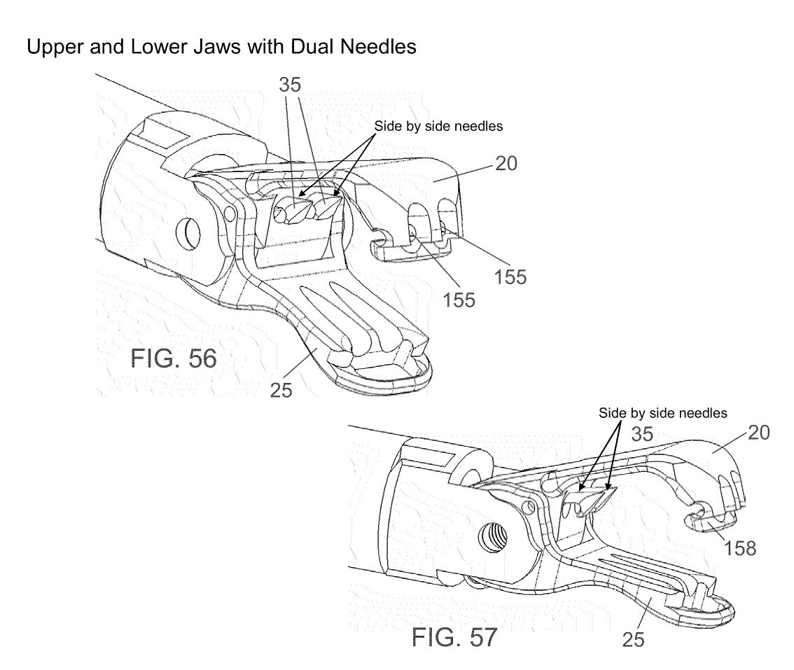

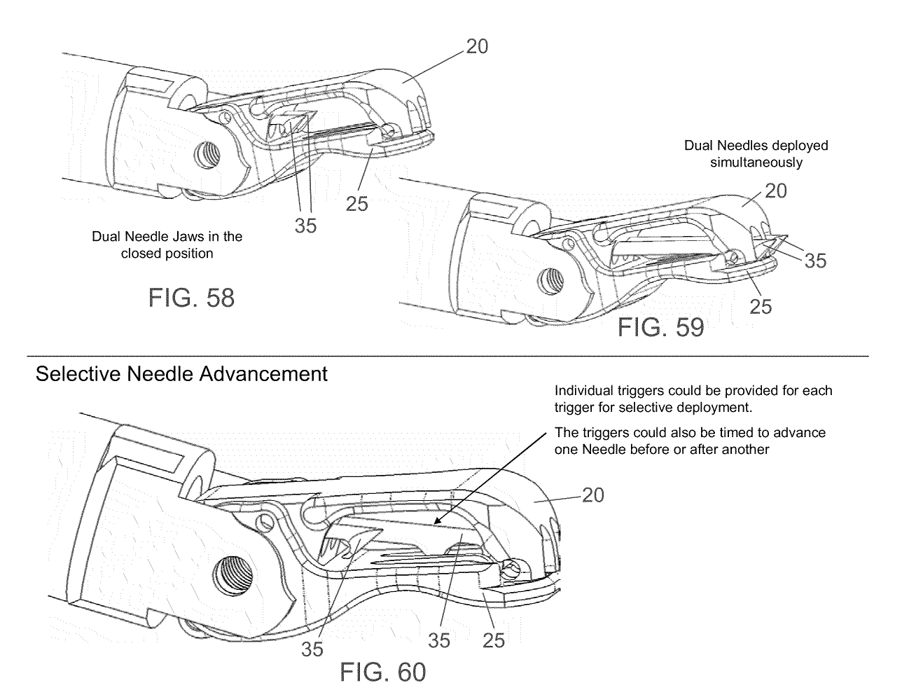

It is also possible to configure suture passer 5 so that it passes multiple needles (and hence multiple suture strands) through the labrum. Thus, for example, and looking now at FIGS. 56-59, two needles 35 may be provided. These needles may be activated together in a coordinated fashion (i.e., extended together and retracted together) with a single mechanism, or the needles may be activated separately (FIG. 60) using separate mechanisms. These multiple needle configurations can enable more beneficial stitches such as a horizontal stitch meant to engage more fibers of the labrum and make a stronger attachment, or to attach multiple suture strands at relatively close locations in the labrum.

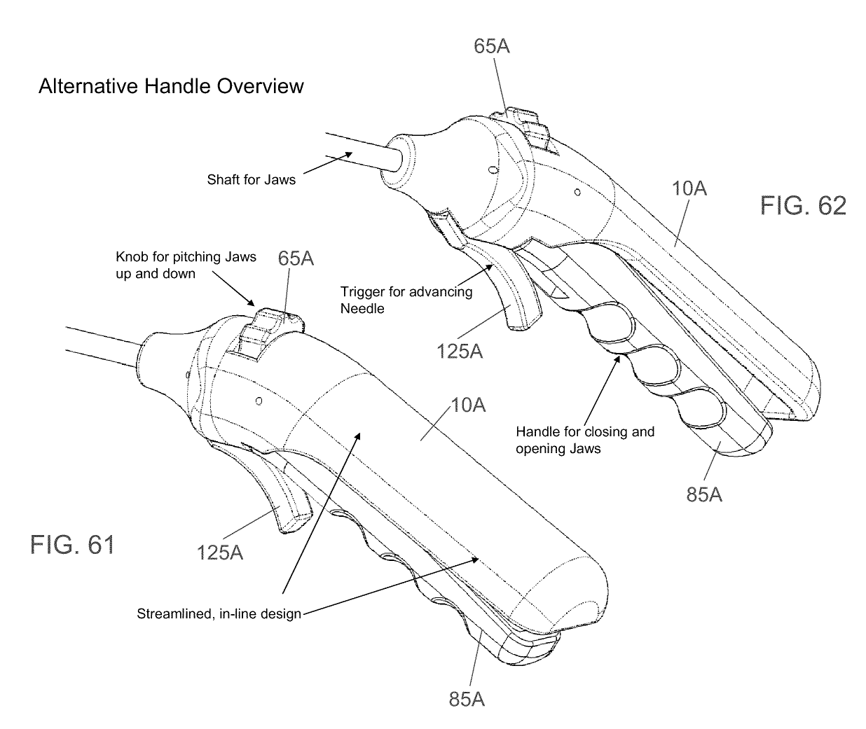

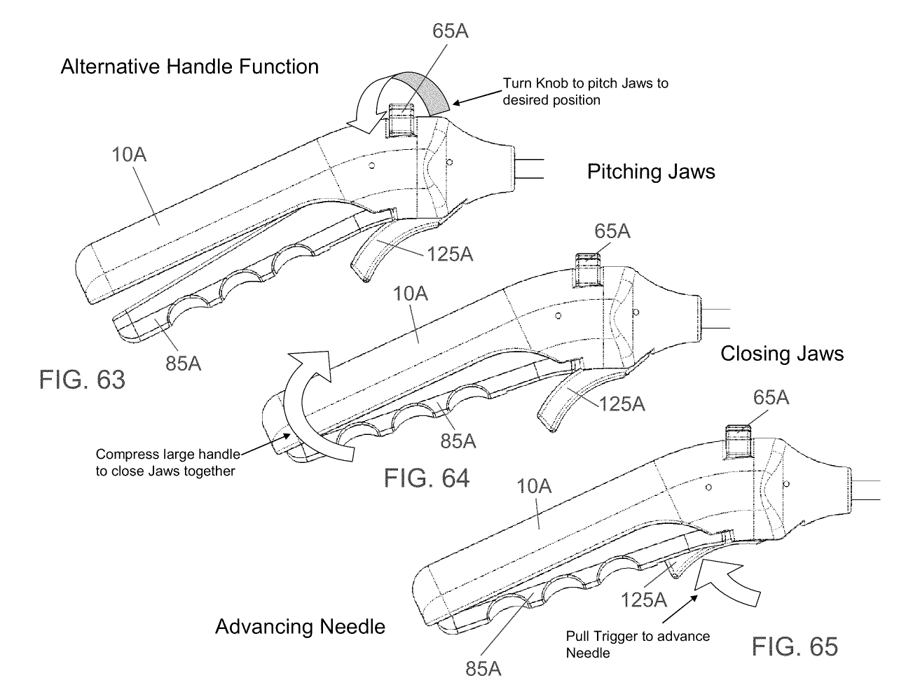

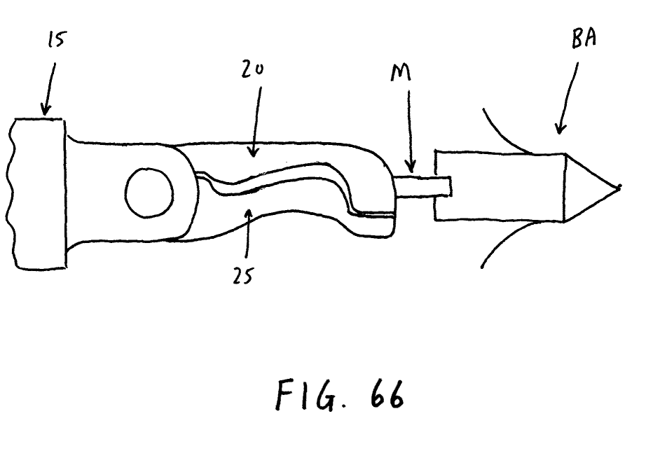

It is also possible to provide suture passer 5 with a different handle mechanism. By way of example but not limitation, and looking now at FIGS. 61-65, there is shown an alternative handle mechanism which comprises a handle 10A, a knob 65A for adjusting the pitch of the jaw members, a lever arm 85A for opening and closing the jaw members, and a trigger 125A for deploying the needle. The “in-line” handle mechanism shown in FIGS. 61-65 can have certain advantages over the “pistol grip” handle mechanism previously disclosed, depending on the surgery being performed. By way of example but not limitation, an in-line handle mechanism may be advantageous where the surgeon is working with his/her hands at his/her waist, which frequently necessitates holding the suture passer with a stabbing posture, or in a surgery where many instruments may be simultaneously disposed around the suture passer, so that the inline handle mechanism provides a streamlined profile which is “out of the way” of the other instruments).

### Additional Constructions

It is also possible to make other changes to suture passer 5 without departing from the scope of the present invention.

Thus, for example, suture passer 5 can be constructed so that its shaft and jaw members can rotate relative to its handle mechanism. This design can be advantageous, e.g., where numerous other instruments are being used in a procedure, the surgeon can use this feature to rotate the handle mechanism away from any other instruments disposed nearby, thereby reducing instrument “collisions”.

Or the handle mechanism may be made so that it is removable from the shaft. This might be done to reduce the aforementioned instrument collisions about a crowded surgical site, or to reduce the weight on the proximal end of the shaft. With this construction, the ratchet mechanism could be located on the shaft (rather than on the handle mechanism), such that when the handle mechanism is removed, the jaw members can still be held in their clamped position.

It is also anticipated that suture passer 5 can be configured so that closing of the jaw members and passing of the needle can be effected by a single mechanism. By way of example but not limitation, the handle mechanism can be configured so that when lever arm 85 is moved a certain distance, the jaw members will be closed, and further pulling of the lever arm will cause the needle to be advanced. Furthermore, a stop or detent feature may be provided to separate the two actions (i.e., between closing the jaw members and passing the needle) so as to give the surgeon a tactile indication as to when the suture passer is transitioning from jaw closure to needle advancement.

It is also possible to vary the angle of motion of the needle within the two jaw members (i.e., the needle may be pitched up or down within the jaws). More particularly, and as discussed above, the axis of needle 35 is generally intended to be set substantially parallel to the axis of shaft 15. However, if desired, it is also possible to change the angle of motion of the needle relative to the axis of the clamped jaw members, e.g., so that the needle could be pitched a few degrees off axis within the jaw members in order to accommodate different surgeries and/or stitch placements within the labrum.

And in another form of the invention, suture passer 5 can be configured to push the suture through the labrum, rather than pulling the suture through the labrum. In other words, in the same way that the “crotchet hook” is used to “drawn” the suture through the tissue, the hook can be configured and used to “push” the suture through the labrum. Furthermore, by combining pulling and pushing of the suture, a mattress stitch can be easily achieved. The mechanisms for pulling and pushing the suture can be formed separate from one another, or they can be formed as a single unit.

In still another form of the invention, and looking now at FIG. 66, jaw members 20, 25 can be configured to hold a bone anchor BA at the distal end of the suture passer. As a result, suture passer 5 can be used to place the suture anchor in a hole prepared in the acetabulum, and then the jaw members could release the anchor. By way of example but not limitation, upper jaw member 20 can be provided with a mount M for releasably holding bone anchor BA to the suture passer. The suture passer could then be used as described above for manipulating the labrum and passing suture therethrough. The advantage of this construction is that the suture passer can also be used as the inserter for the suture anchor, thereby eliminating additional tools and surgical steps, which results in added convenience for the surgeon.

### Use of the Suture Passer for Other Applications

It should be appreciated that suture passer 5 may also be used for passing suture through other soft tissue of the hip joint, or for passing suture through soft tissue of other joints, or for passing suture through soft tissue elsewhere in the body.

### MODIFICATIONS OF THE PREFERRED EMBODIMENTS

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A suture passer comprising:
a handle;
a shaft extending distally from the handle;
first and second jaw members mounted to the distal end of the shaft, the first jaw member having a suture support for supporting a length of suture;
a pitch adjustment mechanism for adjusting the pitch of the first and second jaw members relative to the shaft;
a lever mechanism for opening and closing the first and second jaw members relative to one another; and
a needle mechanism for selectively urging a needle having a groove therein so that the groove in the needle can engage a length of suture supported by the suture support.

2. A suture passer according to claim 1 wherein the first and second jaw members are contoured so as to reconfigure tissue captured between the first and second jaw members.

3. A suture passer according to claim 1 wherein the first and second jaw members are selectively detachable from the shaft.

4. A suture passer according to claim 1 wherein the suture support is formed integral with the first jaw.

5. A suture passer according to claim 1 wherein the suture support slidably supports a length of suture.

6. A suture passer according to claim 5 wherein the suture support comprises a groove for slidable receiving a length of suture therein.

7. A suture passer according to claim 1 wherein the suture support faces substantially distally.

8. A suture passer according to claim 1 wherein the suture support faces substantially proximally.

9. A suture passer according to claim 1 wherein the pitch adjustment mechanism is operated by a knob rotatably mounted on the handle.

10. A suture passer according to claim 1 wherein the needle mechanism is configured to urge the needle substantially axial to the shaft.

11. A suture passer according to claim 1 wherein the needle mechanism is configured to urge the needle substantially axial to the first jaw member, regardless of the pitch of the first jaw member relative to the shaft.

12. A suture passer according to claim 1 wherein the needle is flexible.

13. A suture passer according to claim 12 wherein the needle flexes when it engages a length of suture supported by the suture support.

14. A suture passer according to claim 1 wherein the needle mechanism is configured so that (i) the needle mechanism can move the needle when the first and second jaw members are in their closed configuration, and (ii) the needle mechanism is prevented from moving the needle when the first and second jaw members are in their open configuration.

15. A suture passer according to claim 1 wherein a bone anchor is releasably mounted to at least one of the first jaw member and the second jaw member.

16. A suture passer according to claim 1 wherein the suture passer is configured to pass suture through labrum on the capsular side of the labrum.

17. A method for passing suture through soft tissue, the method comprising the steps of:
providing a suture passer comprising:
a handle;
a shaft extending distally from the handle;
first and second jaw members mounted to the distal end of the shaft, the first jaw member having a suture support for supporting a length of suture;
a pitch adjustment mechanism for adjusting the pitch of the first and second jaw members relative to the shaft;
a lever mechanism for opening and closing the first and second jaw members relative to one another; and
a needle mechanism for selectively urging a needle having a groove therein so that the groove in the needle can engage a length of suture supported by the suture support;
positioning the needle so that it is withdrawn from the opening, positioning the first and second jaw members so that they are in their closed position, and positioning the suture in the suture support;
advancing the suture passer so that the first and second jaw members are adjacent to the tissue through which the suture is to be passed;
opening the first and second jaw members, adjusting the pitch of the first and second jaw members so that the first and second jaw members are aligned with the tissue, advancing the suture passer so that the first and second jaw members engage the tissue, and closing the first and second jaw members so that they securely grasp the tissue;
advancing the needle so that it passes through the tissue and the groove engages the suture; and
withdrawing the needle so that it passes out of the tissue, carrying the suture therewith, so that the suture is passed through the tissue.

18. A method according to claim 17 wherein the tissue is repositioned prior to advancing the needle.

19. A method according to claim 17 wherein the tissue comprises the labrum.

20. A method according to claim 17 wherein the suture is passed through the labrum on the capsular side of the labrum.

21. A suture passer comprising:
a handle;
a shaft extending distally from the handle;
first and second jaw members pivotally mounted to the distal end of the shaft, the first jaw member having an opening formed therein and a suture support for supporting a length of suture adjacent the opening;
a slide movably mounted to the handle;
a lever arm for moving the slide relative to the handle;
first and second yokes movably disposed on the slide, and a yoke movement mechanism for selectively (i) urging first yoke distally and second yoke proximally, or (ii) urging first yoke proximally and second yoke distally;
a first control rod for connecting the first yoke to the first jaw member, and a second control rod for connecting the second yoke to the second jaw member;
a trigger pivotally mounted to the handle;
a needle slidably disposed within the shaft, the distal end of the needle having a groove therein and the proximal end of the needle being secured to the trigger so that the trigger can urge the needle through the opening in the first jaw member so that the groove in the needle can engage a length of suture supported by the suture support;
whereby (i) the pitch of the first and second jaw members relative to the longitudinal axis of the shaft may be adjusted via the yoke movement mechanism, (ii) the first and second jaw members may be opened and closed relative to one another via movement of the lever arm relative to the handle, and (iii) the needle can be advanced through the opening in the first jaw member so that the groove in the needle can engage a length of suture supported by the suture support via movement of the trigger relative to the handle.

22. A suture passer according to claim 21 further comprising a first spring for biasing the lever arm relative to the handle so that the first and second jaw members are biased into their open position.

23. A suture passer according to claim 22 further comprising a ratcheting mechanism for releasably securing the disposition of the lever arm relative to the handle.

24. A suture passer according to claim 21 further comprising a second spring for biasing the trigger relative to the handle so that the needle is biased away from the opening.

25. A suture passer according to claim 21 wherein the needle is flexible.

26. A method for passing suture through soft tissue, the method comprising:
providing a suture passer comprising:
a handle;
a shaft extending distally from the handle;
first and second jaw members pivotally mounted to the distal end of the shaft, the first jaw member having an opening formed therein and a suture support for supporting a length of suture adjacent the opening;
a slide movably mounted to the handle;
a lever arm for moving the slide relative to the handle;
first and second yokes movably disposed on the slide, and a yoke movement mechanism for selectively (i) urging first yoke distally and second yoke proximally, or (ii) urging first yoke proximally and second yoke distally;
a first control rod for connecting the first yoke to the first jaw member, and a second control rod for connecting the second yoke to the second jaw member;
a trigger pivotally mounted to the handle;
a needle slidably disposed within the shaft, the distal end of the needle having a groove therein and the proximal end of the needle being secured to the trigger so that the trigger can urge the needle through the opening in the first jaw member so that the groove in the needle can engage a length of suture supported by the suture support;
whereby (i) the pitch of the first and second jaw members relative to the longitudinal axis of the shaft may be adjusted via the yoke movement mechanism, (ii) the first and second jaw members may be opened and closed relative to one another via movement of the lever arm relative to the handle, and (iii) the needle can be advanced through the opening in the first jaw member so that the groove in the needle can engage a length of suture supported by the suture support via movement of the trigger relative to the handle;
positioning the needle so that it is withdrawn from the opening, positioning the first and second jaw members in their closed position, and positioning the suture in the suture support;
advancing the suture passer so that the first and second jaw members are adjacent to the tissue through which the suture is to be passed;
opening the first and second jaw members, adjusting the pitch of the first and second jaw members so that the first and second jaw members are aligned with the tissue, advancing the suture passer so that the first and second jaw members engulf the tissue, and closing the first and second jaw members;
advancing the needle so that it passes through the tissue and through the opening so that the groove engages the suture; and
withdrawing the needle so that it passes out of the opening and out of the tissue, carrying the suture therewith, so that the suture is passed through the tissue.

27. A method according to claim 26 wherein the tissue is repositioned prior to advancing the needle.

28. A method according to claim 26 wherein the tissue comprises the labrum.

29. A method according to claim 26 wherein the suture is passed through the labrum on the capsular side of the labrum.

30. A suture passer according to claim 1 wherein the lever mechanism is configured to operate regardless of the pitch of the jaw members relative to the shaft.

31. A method according to claim 17 wherein the lever mechanism is configured to operate regardless of the pitch of the jaw members relative to the shaft.
